(19) United States
(12) Patent Application Publication
(10) Pub. No.: US 2026/0315430 A1
(43) Pub. Date: Oct. 15, 2026

(54) CABAZITAXEL MICELLE AND PREPARATION METHOD THEREFOR

(71) Applicants: SHANGHAI WHITTILONG PHARMACEUTICAL INSTITUTE, Shanghai (CN); SHANGHAI WHITTILONG PHARMACEUTICAL LTD, Shanghai (CN)

(72) Inventors: Jing LUO, Shanghai (CN); Yue WANG, Shanghai (CN); Zounian TAN, Shanghai (CN); Xiaodong ZHAO, Shanghai (CN); Hao WANG, Shanghai (CN)

(21) Appl. No.: 19/672,539

(22) Filed: May 8, 2026

Related U.S. Application Data
(63) Continuation of application No. PCT/CN2025/108932, filed on Jul. 16, 2025.

(30) Foreign Application Priority Data
Jul. 16, 2024 (CN) 202410952693.8

(51) Int. Cl. A61K 31/337; A61K 9/00; A61K 9/107; A61K 47/02; A61K 47/10; A61K 47/26; A61K 47/34

(52) U.S. Cl. CPC A61K 31/337; A61K 9/0019; A61K 9/1075; A61K 47/02; A61K 47/10; A61K 47/26; A61K 47/34

(57) ABSTRACT

Disclosed are a cabazitaxel micelle and a preparation method thereof. The cabazitaxel micelle of the present invention comprises cabazitaxel and a polyethylene glycol-derived phospholipids. The polyethylene glycol-derived phospholipid comprises a polyethylene glycol moiety and a phospholipid moiety, and the phospholipid moiety is di(C12-C24 fatty acyl)phosphatidylethanolamine. The polydispersity index of the micelle is not greater than 0.09. The cabazitaxel micelle of the present invention has one or more of the following advantages: uniform particle size, good stability, low impurity content, high release rate, short reconstitution time, and convenient clinical administration.

This application claims priority to Chinese Patent Application No. 2024109526938, filed on Jul. 16, 2024, the entire contents of which are incorporated herein by reference.

### TECHNICAL FIELD

The present invention relates to a Cabazitaxel micelle and a preparation method thereof.

Taxanes are a large class of natural drugs obtained from plants and structurally classified as diterpenoids. Both Cabazitaxel and Docetaxel belong to the taxane class and are liposoluble compounds. However, their clinical application is limited due to their poor water solubility, short half-life, and high toxicity. Currently, the marketed Docetaxel and Cabazitaxel injection solutions both dissolve the drugs in Tween-80. In clinical use, they must be strictly diluted with a dedicated injection solvent, and the operation requirements are stringent. Special infusion devices are also required during infusion. Not only is the usage method cumbersome, but the large amount of Tween-80 in the formulation can also easily cause hemolysis and allergic reactions, etc. Dexamethasone and other drugs need to be taken in advance for prevention. The clinical medication is inconvenient and safety profile is poor (Annals of Allergy, Asthma and Immunology, 2005, 95(6): 593-599. Journal of Investigational Allergology and Clinical Immunology, 2016, 26, 394-396). So far, this problem has not been well solved.

There are already taxane-containing liposomes in the prior art, yet they suffer many disadvantages. For example, the preparation process of liposomes is complex, requiring the combination of multiple lipid components (at least two lipid components), and the particle size control requires special equipment and devices. They are prone to flocculation during storage, etc.

The encapsulation efficiency of Cabazitaxel liposomes reported by Li Zhang et al. from Jiangsu University is only about 86%, and the particle size changes significantly before and after freeze-drying, increasing from 68 nm to 108 nm, which leads to increased instability of the liposomes upon reconstitution. (Zhang Li. Preparation and Pharmacokinetic Study of Cabazitaxel Lyophilized Liposomes [D]. Jiangsu: Jiangsu University, 2017.)

long-circulating liposomes can be prepared by adding a certain proportion of polyethylene glycol-derivatized phospholipids to a conventional liposome formulation. The long PEG chains form a steric hindrance layer and a hydrophilic protective layer on the surface of the liposomes, which can prevent recognition and uptake by phagocytes, increase their circulation time in the blood, and endow this type of liposomes with long-circulating properties. However, when the mass ratio of polyethylene glycol-derivatized phospholipids to phospholipids exceeds a certain value, the encapsulation efficiency and stability of the drug in the liposomes significantly decrease. This is because when the content of polyethylene glycol-derivatized phospholipids is high, they are difficult to insert into the phospholipids, thereby affecting the stability of the liposomes. (Colloids and Surfaces B: Biointerfaces 155(2017): 266-275.)

Polymer nanomicelles are drug delivery systems, with a core-shell structure developed in recent years for poorly soluble drugs. The hydrophobic core and hydrophilic allow poorly soluble drugs to be loaded in the core thereby achieving solubilization. Compared with commonly used solubilizers such as Tween-80 and polyoxyethylated castor oil, polymer micelle carriers are often made of biodegradable materials with high safety orifiles and therefore have a good application prospect as carrier excipients for poorly soluble drugs (Journal of Controlled Release, 2001, 73, 137-172. Pharmaceutical Research, 2007, 24(1), 1-16). At present, there are many nanoformulations of taxane drugs under development (CN103990135A. CN107625730A. CN104758256A). these have, to some extent, addressed the issues of poor water solubility, adverse reactions and low bioavailability of taxane drugs. However, current nanoformulations of taxane drugs suffer from issues such as low drug loading and encapsulation efficiency, and poor stability (CN103990135A. CN107625730A. CN104758256A. CN102293736A. CN101732234A. CN101804021A. CN101829046A). The drug loading of existing nanoformulations is generally less than 5%, and the encapsulation efficiency is less than 90%, which leads to the inclusion of a large amount of carrier materials in the formulation. Since many nanomaterials have not been verified for safety in clinical use, there is a risk of generating toxic substances during degradation in the human body, which limits their clinical application (Journal of Controlled Release, 2009, 133, 11-17. International Journal of Pharmaceutics, 2014, 464(1-2): 178-184). On the other hand, most nanoformulations of poorly soluble drugs have poor stability, and the encapsulation efficiency decrease within a short time, leading to drug leakage. Especially for taxane drugs, when the encapsulation efficiency drops below 95%, insoluble particles appear in the drug solution, affecting the quality of the formulation and preventing its practical clinical application. (CN103990135A, CN107625730A, CN104758256A). At the same time, many nanoformulations have complex preparation processes, and some even require heating and stirring steps, which pose risks for clinical application (CN101972480A. Drug Delivery and Translational Research, 2018, 5, 1365-1379).

Polyethylene glycol (PEG)-derivatized phospholipids, such as PEG-DSPE, have a series of unique advantages when used as a micellar drug delivery system. PEG-DSPE os a drug excipient approved by the FDA with good safety profile (Journal of Controlled Release, 2013, 171, 133-142. Pharmaceutical Research, 2012, 29, 1977-1989. Journal of the National Cancer Institute, 2007, 99, 1004-1015). Our previous research has shown that certain water-soluble small molecule drugs can be successfully loaded into PEG-DSPE nanomicelles through a simple one-step self-assembly process in aqueous solution (Pharmaceutical Research, 2010, 27(2), 361-370). These drug-loaded micelles unload their payloads into the cell membrane by disassembling and inserting into it, thereby increasing membrane fluidity and accelerating intracellular drug transport (Journal of Controlled Release, 2012, 160, 637-651). Meanwhile, we recently discovered that PEG-DSPE nanomicelles possess function similar to natural molecular chaperones. The hydrophilic nanocages formed by their shells can accommodate the A and B chains of insulin, promoting correct folding of the two chains and preventing insulin aggregation. Based on the spatial structure of the nanocages, PEG-DSPE nanomicelles can accommodate protein molecules with a molecular weight of less than 20 KD (Biomaterials, 2016, 77, 139-148). Although PEG-DSPE has excellent drug loading capacity, it exhibits structural selectivity toward the drugs it carries. For instance, doxorubicin hydrochloride, vinorelbine tartrate, and vincristine sulfate can be successfully loaded into PEG-DSPE micelles with an encapsulation efficiency as high as 99.9% (CN1840193A. CN101322681A. CN1739525A. CN101138545A). However, PEG-DSPE micelles cannot be successfully and stably encapsulated PEG-DSPE micelles, thus failing to meet clinical requirement. (Pharmaceutical Research, 2010, 27(2), 361-370.) Philosophical Transactions of The Royal Society A, 20120309, 371. Patent CN113908123A discloses a micelle containing taxane drugs, a preparation method thereof, and an application thereof. In this patent, a film hydration method is used to prepare Cabazitaxel micelles. In this method, Cabazitaxel and the micelle-forming material PEG-PE are dissolved in an organic solvent, and then the organic solution containing the drug is heated and rotary-evaporated to remove the organic solvent. This method has the disadvantages of high energy consumption, adverse effects of heating on drug stability, small batch size of the drug produced by the rotary evaporation method, and being unfavorable for commercial-scale production. Moreover, the Cabazitaxel micelles prepared by this method have non-uniform particle size, poor stability, high impurity content, low drug release rate, long reconstitution time, and poor convenience for clinical use.

### Invention Summary

The technical problem to be solved by the present invention is to overcome the defects in the prior art preparation methods for taxane-containing micelles, such as high energy consumption, heating affecting drug stability, and small batch sizes produced by rotary evaporation, which is unfavorable for commercial scale-up, as well as the defects of the obtained Cabazitaxel micelles, such as non-uniform particle sizes, poor stability, high impurity content, low release rate, long reconstitution time and poor clinical convenience. Therefore, the present invention provides a method for preparing Cabazitaxel micelle, which has one or more of the following advantages: low energy consumption, no requirement for high temperatures, capability for batch production, facilitating commercial scale-up, uniform particle sizes of the product, good stability, low impurity content, high drug release rate, short reconstitution time and good convenience for clinical use.

The present invention provides Cabazitaxel-containing micelle, which comprise Cabazitaxel and polyethylene glycol-derivatized phospholipids.

The polyethylene glycol-derivatized phospholipids comprise a polyethylene glycol moiety and a phospholipid moiety, and the phospholipid moiety is di(C₁₂-C₂₄ fatty acyl) phosphatidylethanolamine.

The polydispersity index (PDI) of the micelle is no more than 0.09.

In some preferred embodiments of the present invention, the materials, ratios and properties of the Cabazitaxel-containing micelle are defined as follows, and the materials, ratios or properties not mentioned are the same as those in any embodiment of the present invention (referred to as “in a certain embodiment of the present invention”). For other embodiments of the present invention, the same abbreviation method as this embodiment is adopted, and no further description is given. In a certain embodiment of the present invention, the Cabazitaxel-containing micelle further include one or more of the following components: C₁₋₆ alcohol solvents (such as ethanol), water (such as sterilized water for injection), glucose injection (such as 5% glucose injection) and sodium chloride injection (such as 0.9% sodium chloride injection).

In a certain embodiment of the present invention, the phospholipid moiety of the polyethylene glycol-derivatized phospholipids may be di-stearoyl phosphatidylethanolamine (C₁₈, abbreviated as DSPE), di-palmitoyl phosphatidylethanolamine (C₁₆, abbreviated as DPPE), di-myristoyl phosphatidylethanolamine (C₁₄, abbreviated as DMPE) or di-oleoyl phosphatidylethanolamine (C₁₈, abbreviated as DOPE), preferably di-stearoyl phosphatidylethanolamine (DSPE).

In a certain embodiment of the present invention, the polyethylene glycol moiety of the polyethylene glycol-derivatized phospholipids has a weight-average molecular weight range of 1000 to 5000, for example, a weight-average molecular weight of 2000.

In a certain embodiment of the present invention, the polyethylene glycol-derivatized phospholipids are polyethylene glycol 1000 di-stearoyl phosphatidylethanolamine (PEG1000-DSPE), polyethylene glycol 2000 di-stearoyl phosphatidylethanolamine (PEG2000-DSPE), polyethylene glycol 5000 di-stearoyl phosphatidylethanolamine (PEG5000-DSPE), polyethylene glycol 2000 di-oleoyl phosphatidylethanolamine (PEG2000-DOPE), polyethylene glycol 2000 di-palmitoyl phosphatidylethanolamine (PEG2000-DPPE) or polyethylene glycol 2000 di-myristoyl phosphatidylethanolamine (PEG2000-DMPE), preferably PEG2000-DSPE.

In a certain embodiment of the present invention, the polydispersity index of the Cabazitaxel-containing micelle is no more than 0.08, for example, 0.08, 0.07, 0.06 or 0.04.

In a certain embodiment of the present invention, the polydispersity index of the Cabazitaxel-containing micelle is no more than 0.06, for example, 0.04.

In a certain embodiment of the present invention, the Zeta potential of the Cabazitaxel-containing micelle is (−45 mv) to (−60 mv), preferably (−45 mv) to (−50 mv), for example, −48.77 mv. In one embodiment of the present invention, in the Cabazitaxel-containing micelle, the mass ratio of polyethylene glycol-derivatized phospholipids to Cabazitaxel is no more than 15:1, for example, (0.5-1.5): 1 or (5-15): 1, or 1:1, 10:1 or 11:1.

In one embodiment of the present invention, in the Cabazitaxel-containing micelle, the mass ratio of polyethylene glycol-derivatized phospholipids to Cabazitaxel is no more than 15:1, for example, (0.5-1.5): 1 or (9-13): 1, or 1:1, 10:1 or 11:1.

In one embodiment of the present invention, in the Cabazitaxel-containing micelle, the mass ratio of polyethylene glycol-derivatized phospholipids to Cabazitaxel is (8-15): 1, preferably (9-13): 1, for example, 10:1 or 11:1.

In one embodiment of the present invention, the reconstitution time of the Cabazitaxel-containing micelle is no more than 60 minutes, preferably no more than 45 minutes, and more preferably no more than 15 minutes, for example, 10 minutes, 7 minutes or 5 minutes.

In one embodiment of the present invention, the encapsulation efficiency of the Cabazitaxel-containing micelle is 95%-100%, preferably 98.5%-100%, for example, 99.70%, 98.90% or 99.90%.

In one embodiment of the present invention, the content of total impurities in the Cabazitaxel-containing micelle is no more than 2%, preferably no more than 1%, and more preferably no more than 0.5%, for example, 0.36% or 0.06%.

In one embodiment of the present invention, the content of total impurities in the Cabazitaxel-containing micelle is no more than 0.1%, for example, 0.06%.

In one embodiment of the present invention, the Cabazitaxel-containing micelle can be stably stored at room temperature for more than 6 hours.

In one embodiment of the present invention, the Cabazitaxel-containing micelle can be stably stored at 4° C. for more than 24 hours.

In one embodiment of the present invention, the drug release of the Cabazitaxel-containing micelle is 75%-100%, preferably 75%-85%, for example, 78.3%.

In one embodiment of the present invention, the particle size of the Cabazitaxel-containing micelle is 10-20 nm, for example, 13 nm, 14 nm, 15 nm or 16 nm.

In one embodiment of the present invention, the Cabazitaxel-containing micelle also contain a lyoprotectant. The lyoprotectant is preferably sucrose or lactose.

In one embodiment of the present invention, the Cabazitaxel-containing micelle comprise Cabazitaxel and polyethylene glycol 2000 distearoyl phosphatidylethanolamine. The mass ratio of Cabazitaxel to polyethylene glycol 2000 distearoyl phosphatidylethanolamine is preferably 1:11. The reconstitution time of the Cabazitaxel-containing micelle is preferably no more than 60 minutes. The encapsulation efficiency of the Cabazitaxel-containing micelle is preferably 95%-100%. The content of total impurities in the Cabazitaxel-containing micelle is preferably no more than 2%. The drug release of the Cabazitaxel-containing micelle is preferably 75%-100%. The particle size of the Cabazitaxel-containing micelle is preferably 10-20 nm. The Zeta potential of the Cabazitaxel-containing micelle is preferably (−45 mv) to (−60 mv).

Preferably, the Cabazitaxel-containing micelle consists of Cabazitaxel and polyethylene glycol 2000 distearoyl phosphatidylethanolamine.

In one embodiment of the present invention, the Cabazitaxel-containing micelle is micelle for treating cancer. Preferably, the cancer is selected from prostate cancer, breast cancer, ovarian cancer, non-small cell lung cancer, head and neck cancer, pancreatic cancer, small cell lung cancer, gastric cancer, melanoma and soft tissue sarcoma.

The present invention also provides a method for preparing Cabazitaxel-containing micelle, which includes the following step A:

The solution of Cabazitaxel and polyethylene glycol (PEG)-derived phospholipids in a polar organic solvent A is subjected to spray drying to obtain micelle A.

The PEG-derived phospholipids include a PEG part and a phospholipid moiety, with the phospholipid part being di(C₁₂-C₂₄ fatty acyl) phosphatidylethanolamine.

The polar organic solvent A has a boiling point below 85° C. and the solubility of Cabazitaxel in solvent A is at least 60 mg/mL at room temperature.

In one embodiment of the invention, the solubility of PEG-derived phospholipids in solvent A is at least 60 mg/mL at room temperature.

In one embodiment of the invention, the polar organic solvent A is acetone, dichloroethane, chloroform, tetrahydrofuran, carbon tetrachloride, ethyl acetate, methyl ethyl ketone or an alcohol solvent.

In one embodiment of the invention, the polar organic solvent A is an alcohol solvent, preferably a C₁₋₆ alcohol solvent, more preferably a C₁₋₃ alcohol solvent, such as ethanol.

In one embodiment of the invention, the phospholipid moiety of the PEG-derived phospholipids may be one or more of distearoyl phosphatidylethanolamine (C₁₈, abbreviated as DSPE), dipalmitoyl phosphatidylethanolamine (C₁₆, abbreviated as DPPE), dimyristoyl phosphatidylethanolamine (C₁₄, abbreviated as DMPE), and dioleoyl phosphatidylethanolamine (C₁₈, abbreviated as DOPE), preferably DSPE.

In one embodiment of the invention, the PEG in the PEG-derived phospholipids has a weight-average molecular weight in the range of 1000 to 5000, for example, 2000.

In one embodiment of the invention, the PEG-derived phospholipids are PEG1000-DSPE, PEG2000-DSPE, PEG5000-DSPE, PEG2000-DOPE, PEG2000-DPPE, or PEG2000-DMPE, preferably PEG2000-DSPE.

In one embodiment of the invention, the mass ratio of the PEG-derived phospholipids to Cabazitaxel in step A is (0.5-1.5): 1, for example, 1:1.

In one embodiment of the invention, the concentration of Cabazitaxel in the polar organic solvent A is 3-80 mg/mL, preferably 6-60 mg/mL, more preferably 30-60 mg/mL, for example, 60 mg/mL.

In one embodiment of the invention, the temperature of the polar organic solvent A solution is 20-60° C., preferably 40° C.

In one embodiment of the invention, the inlet air temperature in spray drying is 45-65° C., preferably 50-60° C., for example, 55° C.

In one embodiment of the invention, the outlet air temperature in spray drying is 30-45° C., preferably 35-45° C., for example, 40° C.

In one embodiment of the invention, the pump speed in spray drying is 5%-15%, preferably 5%-10%, for example, 8%.

In one embodiment of the invention, the air speed in spray drying is 40%-80%, preferably 45%-60%, for example, 50%.

In one embodiment of the invention, the nozzle cleaning frequency in spray drying is 3-5 times/min, for example, 4 times/min.

In one embodiment of the present invention, the preparation method of the Cabazitaxel-containing micelle further comprises the following step B:
Dissolving the micelle A and polyethylene glycol-derivatized phospholipids in water to obtain micelle B;
preferably, the water is sterile water for injection;
the ratio of the mass of Cabazitaxel contained in the micelle A to the volume of the water is 2-10 mg/mL, preferably 4-8 mg/mL, for example, 6 mg/mL.

In one embodiment of the present invention, the concentration of the polyethylene glycol-derivatized phospholipids in the water in step B is 40-80 mg/mL, for example, 60 mg/mL or 66 mg/mL.

In one embodiment of the present invention, the mass ratio of the polyethylene glycol-derivatized phospholipids to the Cabazitaxel contained in the micelle A in step B is (5-15): 1, for example, 10:1 or 11:1.

In one embodiment of the present invention, the mass ratio of the polyethylene glycol-derivatized phospholipids to the Cabazitaxel in step B is (8-15): 1, preferably (9-13): 1, for example, 10:1 or 11:1.

In one embodiment of the present invention, step B is carried out at 15-30° C., for example, 25° C.

In one embodiment of the present invention, the preparation method of the Cabazitaxel-containing micelle further comprises the following step C:

Filtering the micelle B to obtain micelle C.

In one embodiment of the present invention, filtration in step C is performed by using a high-molecular-weight filter membrane.

Preferably, the high-molecular-weight filter membrane is a polyvinylidene fluoride (PVDF) membrane, a mixed cellulose ester (MCE) membrane, a polyethersulfone (PES) membrane, a nylon membrane, a polytetrafluoroethylene (PTFE) membrane, or a polypropylene (PP) membrane.

Preferably, the pore size of the polyvinylidene fluoride (PVDF) membrane is 0.10-0.30 mm, for example, 0.22 mm.

In one embodiment of the present invention, step C is carried out at 15-30° C., for example, 25° C.

In one embodiment of the present invention, the preparation method of the Cabazitaxel-containing micelle further comprises the following step D:

Removing the water from the micelle C to obtain micelle D.

In one embodiment of the present invention, the water in the micelle C in step D is removed by freeze-drying, spray-drying, or fluidized drying.

In one embodiment of the present invention, the freeze-drying is carried out in the presence of alyoprotectant, preferably the lyoprotectant is sucrose or lactose; preferably the amount of the lyoprotectant is 2%-6%, for example, 4%; the amount refers to the mass-to-volume ratio of the lyoprotectant to the micelle C.

In one embodiment of the present invention, the freeze-drying is carried out in the presence of a lyoprotectant, or in the absence of a lyoprotectant.

Preferably, the freeze-drying is carried out in the absence of a lyoprotectant.

In one embodiment of the present invention, the freeze-drying is carried out in a conventional manner, preferably the freeze-drying includes a freezing stage, a primary drying stage, and a secondary drying stage.

In one embodiment of the present invention, the preparation method of the Cabazitaxel-containing micelle further comprises the following step E:

Dissolving the micelle D in a solvent a to obtain micelle E.

Preferably, the solvent a is water (for example, sterile water for injection), glucose injection (for example, 5% glucose injection), sodium chloride injection (for example, 0.9% sodium chloride injection), or a combination thereof.

In one embodiment of the present invention, step E is carried out at 15-30° C., for example, 25° C.

In one embodiment of the present invention, the materials used in steps A, B, C, D, and E consist solely of those specified in each respective step and contain no additional materials.

In one embodiment of the present invention, the preparation method of the Cabazitaxel-containing micelle comprises the following steps:
Step A: The solution of Cabazitaxel and PEGylated phospholipid in polar organic solvent A is spray-dried to obtain micelle A.
Step B: micelle A and PEGylated phospholipid are dissolved in water to obtain micelle B.
Step C: micelle B is filtered to obtain micelle C.
Step D: Water is removed from micelle C to obtain micelle D.
Step E: micelle D is dissolved in solvent a to obtain micelle E.

Wherein the conditions, operations or materials in steps A, B, C, D and E are described in any of the preceding schemes.

Preferably, the preparation method of Cabazitaxel-containing micelle consists of steps A, B, C, D and E.

In one embodiment of the present invention, the preparation method of Cabazitaxel-containing micelle is carried out as follows: Cabazitaxel and PEG2000-DSPE are weighed and dissolved in polar organic solvent A to obtain the corresponding solution, which is spray-dried to obtain micelle A; micelle A and PEG2000-DSPE are dissolved in water to obtain micelle B; micelle B is filtered to obtain micelle C; water in micelle C is removed by freeze-drying to obtain micelle D; micelle D is dissolved in solvent a to obtain micelle E; preferably the polar organic solvent A is ethanol; preferably the solvent a is water.

Wherein the conditions, operations or materials not specifically mentioned are as described in any of the preceding schemes.

The present invention also provides Cabazitaxel-containing micelle, which are micelle A, micelle B, micelle C, micelle D or micelle E.
Micelle A is obtained by step A.
Micelle B is obtained by steps A and B.
Micelle C is obtained by steps A, B and C.
Micelle D is obtained by steps A, B, C and D.
Micelle E is obtained by steps A, B, C, D and E.

Wherein the conditions, operations or materials in steps A, B, C, D and E are described in any of the preceding schemes.

In one embodiment of the present invention, the reconstitution time of micelle D is no more than 60 minutes, preferably no more than 45 minutes, more preferably no more than 15 minutes, for example, 10 minutes, 7 minutes or 5 minutes.

In one embodiment of the present invention, micelle E is clear and transparent solution.

In one embodiment of the present invention, the polydispersity index of micelle E is no more than 0.09, preferably no more than 0.08, for example, 0.08, 0.07, 0.06 or 0.04.

In one embodiment of the present invention, the polydispersity index of micelle E is no more than 0.06, for example, 0.04.

In one embodiment of the present invention, the encapsulation efficiency of micelle E is 95%-100%, preferably 98.5%-100%, for example, 99.70%, 98.90% or 99.90%.

In one embodiment of the present invention, the Zeta potential of micelle E or micelle C is (−45 mv)-(−60 mv), preferably (−45 mv)-(−50 mv), for example, −48.77 mv.

In one embodiment of the present invention, the content of total impurities in micelle E is no more than 2%, preferably no more than 1%, more preferably no more than 0.5%, for example, 0.36% or 0.06%.

In one embodiment of the present invention, the content of total impurities in micelle E is no more than 0.1%.

In one embodiment of the present invention, micelle E can be stably stored at room temperature for more than 6 hours.

In one embodiment of the present invention, micelle E can be stably stored at 4° C. for more than 24 hours.

In one embodiment of the present invention, the release rate of micelle E is 75%-100%, preferably 75%-85, for example, 78.3%.

In one embodiment of the present invention, the particle size of the micelle E is 10-20 nm, for example, 13 nm, 14 nm, 15 nm or 16 nm.

In one embodiment of the present invention, the micelles A, B, C, D and E are micelles used for treating cancer.

Preferably, cancer is selected from prostate cancer, breast cancer, ovarian cancer, non-small cell lung cancer, head and neck cancer, pancreatic cancer, small cell lung cancer, gastric cancer, melanoma and soft tissue sarcoma.

The present invention also provides a pharmaceutical composition, which comprises any one of the Cabazitaxel-containing micelle, micelle A, micelle B, micelle C, micelle D or micelle E as described above, and a pharmaceutically acceptable excipient.

The present invention also provides the use of any one of the Cabazitaxel-containing micelle, micelle A, micelle B, micelle C, micelle D, micelle E, or the aforementioned pharmaceutical composition, for the preparation of a medicament for the treatment of cancer.

In one embodiment of the present invention, cancer is selected from breast cancer, ovarian cancer, non-small cell lung cancer, head and neck cancer, pancreatic cancer, small cell lung cancer, gastric cancer, melanoma and soft tissue sarcoma.

In another embodiment of the present invention, the cancer is selected from prostate cancer, breast cancer, ovarian cancer, non-small cell lung cancer, head and neck cancer, pancreatic cancer, small cell lung cancer, gastric cancer, melanoma and soft tissue sarcoma.

The present invention also provides a method for treating cancer in a subject in need thereof, comprising administering to the subject a substance X, wherein said substance X is any one of the Cabazitaxel-containing micelle, micelle A, micelle B, micelle C, micelle D, micelle E, or the aforementioned pharmaceutical composition.

Cancer is selected from prostate cancer, breast cancer, ovarian cancer, non-small cell lung cancer, head and neck cancer, pancreatic cancer, small cell lung cancer, gastric cancer, melanoma and soft tissue sarcoma.

The present invention also provides a substance X for treating cancer.

The substance X is any one of the Cabazitaxel-containing micelle, micelle A, micelle B, micelle C, micelle D, micelle E, or the aforementioned pharmaceutical composition.

Cancer is selected from prostate cancer, breast cancer, ovarian cancer, non-small cell lung cancer, head and neck cancer, pancreatic cancer, small cell lung cancer, gastric cancer, melanoma and soft tissue sarcoma.

Unless otherwise specified, the terms used in the present invention may have the following definitions:
The “micelle” or “micelles” as described in the present invention can be in solid form or liquid form.
The “total impurities” as described in the present invention refers to the cumulative value of all impurities in the substance to be tested that are greater than the reporting limit.

Based on the common sense in the field, the above preferred conditions can be combined arbitrarily to obtain various embodiments of the present invention.

The reagents and raw materials used in the present invention are all commercially available.

The beneficial effects of the present invention reside in that the preparation method of Cabazitaxel micelle provided by the present invention has one or more of the following advantages: low energy consumption, no requirement for high temperature, capability for batch production, facilitating commercial scale-up, uniform particle size, good stability, low impurity content, high drug release rate, short reconstitution time and good convenience for clinical use.

### DRAWING

FIG. 1 shows the transmission electron microscopy (TEM) appearance of the micelle prepared by the film hydration method in Example 1.

FIG. 2 shows the TEM appearance of the micelle prepared by the spray drying method in Example 1.

FIG. 3 shows the appearance of the aqueous solution of the micelle formulation prepared in Example 2 when the ratio of API to palmitoyl phosphatidylethanolamine is 1:7 after hydration.

FIG. 4 shows the appearance of the aqueous solution of the micelle formulation prepared in Example 2 when the ratio of API to palmitoyl phosphatidylethanolamine is 1:11 after hydration.

FIG. 5 shows the appearance of the aqueous solution of the micelle formulation prepared in Example 2 when the ratio of API to palmitoyl phosphatidylethanolamine is 1:15 after hydration.

FIG. 6 shows the appearance of the aqueous solution of the micelle formulation prepared without a lyoprotectant in Example 3 after reconstitution.

FIG. 7 shows the appearance of the aqueous solution of the micelle formulation prepared with sucrose as the lyoprotectant in Example 3 after reconstitution.

FIG. 8 shows the appearance of the aqueous solution of the micelle formulation prepared with lactose as the lyoprotectant in Example 3 after reconstitution.

FIG. 9 shows the appearance of the finished product of the micelle formulation prepared in the absence a lyoprotectant in Example 3.

FIG. 10 shows the appearance of the finished product of the micelle formulation prepared with sucrose as the lyoprotectant in Example 3.

FIG. 11 shows the appearance of the finished product of the micelle formulation prepared with lactose as the lyoprotectant in Example 3.

FIG. 12 shows the growth trend of tumors in each group in Example 6.

FIG. 13 shows the tumor weight inhibition rate in each group in Example 6.

FIG. 14 shows the average drug-time curve of Cabazitaxel in tumors of tumor-bearing mice after intravenous injection of the test Cabazitaxel micelle (i.e., LN003) and the reference solution JEVTANA in Example 7.

FIG. 15 shows the average drug-time curve of Cabazitaxel in the heart of tumor-bearing mice after intravenous injection of the test Cabazitaxel micelle (i.e., LN003) and the reference solution JEVTANA in Example 7.

FIG. 16 shows the average drug-time curve of Cabazitaxel in the liver of tumor-bearing mice after intravenous injection of the test Cabazitaxel micelle (i.e., LN003) and the reference solution JEVTANA in Example 7.

FIG. 17 shows the average drug-time curve of Cabazitaxel in the spleen of tumor-bearing mice after intravenous injection of the test Cabazitaxel micelle (i.e., LN003) and the reference solution JEVTANA in Example 7.

FIG. 18 shows the average drug-time curve of Cabazitaxel in the lungs of tumor-bearing mice after intravenous injection of the test Cabazitaxel micelle (i.e., LN003) and the reference solution JEVTANA in Example 7.

FIG. 19 shows the average drug-time curve of Cabazitaxel in the kidneys of tumor-bearing mice after intravenous injection of the test Cabazitaxel micelle (i.e., LN003) and the reference solution JEVTANA in Example 7.

FIG. 20 shows the average drug-time curve of Cabazitaxel in the brain of tumor-bearing mice after intravenous injection of the test Cabazitaxel micelle (i.e., LN003) and the reference solution JEVTANA in Example 7.

FIG. 21 shows the average drug-time curve of Cabazitaxel in the stomach of tumor-bearing mice after intravenous injection of the test Cabazitaxel micelle (i.e., LN003) and the reference solution JEVTANA in Example 7.

FIG. 22 shows the average drug-time curve of Cabazitaxel in the lymph nodes of tumor-bearing mice after intravenous injection of the test Cabazitaxel micelle (i.e., LN003) and the reference solution JEVTANA in Example 7.

FIG. 23 shows the average drug-time curve of Cabazitaxel in the duodenum of tumor-bearing mice after intravenous injection of the test Cabazitaxel micelle (i.e., LN003) and the reference solution JEVTANA in Example 7.

FIG. 24 shows the average drug-time curve of Cabazitaxel in the testes of tumor-bearing mice after intravenous injection of the test Cabazitaxel micelle (i.e., LN003) and the reference solution JEVTANA in Example 7.

FIG. 25 shows the average drug-time curve of Cabazitaxel in the blood of tumor-bearing mice after intravenous injection of the test Cabazitaxel micelle (i.e., LN003) and the reference product JEVTANA solution in Example 7.

FIG. 26 shows the average drug concentration bar chart of Cabazitaxel in tissues and plasma of tumor-bearing mice after intravenous injection of the test Cabazitaxel micelle (i.e., LN003) in Example 7; each group of data involves data at four time points, and the data bars from left to right represent the average drug concentration at 2 h, 8 h, 24 h and 96h, respectively.

FIG. 27 shows the average drug concentration bar chart of Cabazitaxel in tissues and plasma of tumor-bearing mice after intravenous injection of the reference product JEVTANA solution in Example 7; each group of data involves data at four time points, and the data bars from left to right represent the average drug concentration at 2 h, 8 h, 24 h and 96h, respectively.

FIG. 28 shows the state of the micelle prepared by the film hydration method after 4 hours of release at 37° C. in the dialysis bag.

FIG. 29 shows the state of the micelle prepared by the spray drying method after 4 hours of release at 37° C. in the dialysis bag.

FIG. 30 shows the state of the micelle diluted with different diluents after 8 hours of storage at 37° C.

### EXAMPLES

The following further describes the present invention through examples, but the present invention is not limited thereby to the scope of these examples. Experimental methods in the following examples for which no conditions are specified are carried out under conventional conditions or in accordance with the instructions of commercial products.

### Analysis of Drug-Loaded Micelle Particle Size

The particle size distribution of drug-loaded micelle (micelle hydration solution or re-dissolved micelle) was determined by dynamic light scattering (DLS). The sample was diluted to 1 mg/mL with physiological saline, and 1 mL of the sample was taken to measure the particle size distribution and polydispersity index (PDI) of the drug-loaded micelle using a Malvern Zetasizer Nano-ZS light scattering particle size analyzer. The laser beam wavelength of the instrument was set at 633 nm, and the angle between the incident light and the scattered light beam was 90°. Each sample was measured for 20 cycles, and the measurement temperature was set at 25° C. The measurement results of each formulation were the average of the results of three batches of formulations. The polydispersity index (PDI) indicates the uniformity of particle size, and the smaller the PDI, the more uniform the particles.

### Drug Encapsulation Efficiency and Drug Loading

Took 1 mL of drug-loaded micelle solution, filtered it through a 220 nm microporous membrane, and determined the concentration of drug-loaded micelle in the drug-loaded micelle solution and the filtrate by high-performance liquid chromatography. The chromatographic conditions are as follows:
Ultracentrifugation. High-performance liquid chromatography (Chinese Pharmacopoeia 2020 Edition, Part IV, General Chapter 0512)
Instrument: Agilent/DAD or VWD detector or equivalent instrument
Chromatographic column: ZORBAX Extend-C₁₈ (150×4.6 mm, 3.5 μm)
Mobile phase: Mobile phase A: water; Mobile phase B: acetonitrile.

Time gradient:

| Time (min) | Mobile phase A % | Mobile phase B % |
|---|---|---|
| 0 | 55 | 45 |
| 12.0 | 10 | 90 |
| 14.0 | 10 | 90 |
| 14.1 | 55 | 45 |
| 20.0 | 55 | 45 |

Column temperature: 30° C.
Flow rate: 1.0 ml/min
Detection wavelength: UV 230 nm
Injection volume: 10 μl
Diluent: Acetonitrile

### Solution Preparation

Mobile phase A: Took an appropriate amount of water, and degassed it ultrasonically.
Mobile phase B: Took an appropriate amount of acetonitrile, and degassed it ultrasonically.
Blank solution: Acetonitrile
Reference solution: Took an appropriate amount of Cabazitaxel working reference substance, accurately weighed it, dissolved it in acetonitrile and quantitatively diluted it to prepare a solution containing approximately 36 μg per 1 ml. Two parallel solutions were prepared.
Test solution A: Took the test solution stock solution from the content determination item as the reconstitution stock solution. Accurately measured 1 ml of the reconstitution stock solution, placed it in a 50 ml volumetric flask, diluted it with acetonitrile to the mark, and shaken well.
Test solution B: Took an appropriate amount of the reconstitution stock solution again, centrifuged it at 10,000 rpm for 10 minutes, accurately measured 1 ml of the supernatant, placed it in a 50 ml volumetric flask, diluted it with the diluent to the mark, and shaken well.

### System Applicability

Accurately measured 10 μl of blank solution and 10 μl of reference solution respectively, and injected them into the liquid chromatograph to record the chromatogram. For five consecutive injections of reference solution (STD1), the RSD of the main peak area was ≤2.0% and the RSD of the retention time was ≤1.0%. The main peak recovery rate of reference solution (STD2) was between 98.0% and 102.0%.
Determination: Accurately measured 10 μl of test solution A, 10 μl of test solution B and 10 μl of reference solution respectively, and injected them into the liquid chromatograph to record the chromatogram.
Calculation: Calculated the content of test solution A (S) and test solution B (E) by the external standard method based on the peak area.

$$S \% = \frac{{A}_{spL} \times {W}_{std} \times P \times {V}_{spL}}{{V}_{std} \times {A}_{std} \times B \times N} \times 100 \%$$

$$E \% = \frac{{A}_{spL} \times {W}_{std} \times P \times {V}_{spL}}{{V}_{std} \times {A}_{std} \times B \times N} \times 100 \%$$

Wherein: AspL represents the peak area of each main peak in the test solution A/B. Astd is the average peak area of the main peak in the reference solution for five consecutive injections. Wstd is the weight of the reference substance in the reference solution, in mg. P is the content of the reference substance, %. Vstd is the dilution factor of the reference solution. VspL is the dilution factor of the test solution A/B. B is the specification, 60. N is the quantity, 3.

It should be no less than 98%.

### Example 1

Spray drying method: Accurately weighed 60 mg of Cabazitaxel and 60 mg of PEG2000-DSPE (source: Lipoid), and stirred them to dissolve in 1 mL of anhydrous ethanol at 40° C. to obtain a mixed ethanol solution of the drug and PEG2000-DSPE as the film-forming solution.

| Parameters | Parameters 1 | Parameters 2 | Parameters 3 | Parameters 4 |
|---|---|---|---|---|
| Inlet air temperature (°C) | 45 | 55 | 65 | 75 |
| Outlet air temperature (°C) | 30 | 40 | 45 | 50 |
| Pump speed (%) | 5 | 8 | 15 | 15 |
| Air speed (%) | 40 | 50 | 80 | 90 |
| Nozzle cleaning (times/min) | 3 | 4 | 5 | 5 |
| Appearance of screening results | Powder | Loose powder | Slight melting of | Full fusion of |
| | agglomeration | | powder | the powder |
| | during spray drying | | | |

If the intake air temperature is too low or the pump speed is too high, the solution cannot be dried completely into powder, and the organic solvent cannot be completely removed, resulting in a low powder collection rate. If the intake air temperature is too high, the powder will melt and adhere to the inner wall of the pipeline. If the air speed is too fast or too slow, the yield will decrease and it will also affect the particle size of the powder. The spray drying conditions of parameter 2 were selected.

The API and PEG2000-DSPE complex spray-dried powder was prepared by spray drying (the spray drying parameters were as follows: intake air temperature 55° C., outlet air temperature 40° C., pump speed: 8%).

600 mg of PEG2000-DSPE was dissolved in 10 mL of injection water to prepare a phospholipid aqueous solution. The above spray-dried powder was stirred and dissolved in 10 mL of the phospholipid aqueous solution to prepare a micellar hydration solution. The hydration solution was filtered twice through a 0.22 μm PVDF membrane to obtain the pre-filling solution. The above solution before filling was filled into medium borosilicate glass vials and freeze-dried to obtain the formulation sample. The freeze-drying was carried out according to the following parameters.

| Freeze-drying | | | Rising and falling temperature | |
|---|---|---|---|---|
| process | Set temperature | | rate (°C/min) | Duration (min) |
| Freezing stage | 5 | °C | / | 60 |
| | −40 | °C | 1 | 165 |
| | −20 | °C | 1 | 80 |
| | −40 | °C | 1 | 140 |
| Primary drying stage | −20 | °C | 1 | 500 |
| | −19 | °C | 1 | 960 |
| Desorption drying | 5 | °C | 1 | 505 |
| stage | 6 | °C | 1 | 480 |
| | 25 | °C | 1 | 480 |
| | 26 | °C | 1 | 480 |

Film hydration method: 60 mg of Cabazitaxel and 60 mg of PEG2000-DSPE were dissolved in ethanol. Then, at 50° C., the ethanol solution was rotary evaporated to remove the solvent and form a drug-lipid film. Next, the remaining 600 mg of PEG2000-DSPE was dissolved in water for injection to form an aqueous phase solution. This aqueous phase solution was used to hydrate the drug-lipid film into a micellar aqueous solution. Finally, the micellar aqueous solution was freeze-dried to obtain the finished drug-loaded micelle.

Reconstitution: The lyophilisate obtained by spray drying or film hydration was dissolved in 10 mL of water for injection.

The summary table of the quality results of the micelle (after reconstitution) prepared by film hydration and spray drying was shown in Table 1.

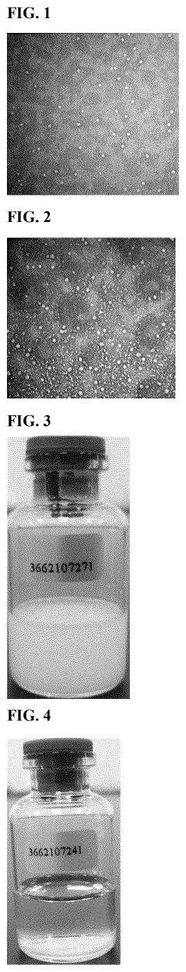

TABLE 1
Summary of Quality Inspection Results of Micelles Prepared
by Film Hydration Method and Spray Drying Method

| | | Film-forming | | | |
|---|---|---|---|---|---|
| Preparation method | | hydration method | | Spray drying method | |
| Reconstitution time | | 120 | min | 10 | min |
| Micellar characteristics (after reconstitution) | | Clear and transparent | | Clear and transparent | |
| | | solution | | solution | |
| Encapsulation efficiency (%) | | 101.5 | | 99.70 | |
| Particle size and | Particle size (nm) | 11.3 | | 15.0 | |
| particle size | Polydispersity index (PDI) | 0.106 | | 0.06 | |
| distribution | | | | | |

| Zeta potential | −44.02 | mv | −48.77 | mv |
|---|---|---|---|---|
| Total impurities (%) | 4.42 | | 0.36 | |
| Transmission electron microscopy appearance | See FIG. 1 | | See FIG. 2 | |
| examination | | | | |

| Micellar solution stability storage time | After being placed at | It is stable for 6 hours |
|---|---|---|
| | 25 °C for 1 hour, a | at room temperature |
| | large amount of | and for 24 hours |
| | crystals precipitated, | at 4 °C |
| | covering the bottom | |
| | of the vial. The | |
| | sample is unstable. | |

| | | |
|---|---|---|
| Drug Release (%) | 70.7 | 78.3 |

The reconstitution time of Cabazitaxel micellar lyophilized powder prepared by spray drying was significantly reduced, which greatly enhanced the convenience of clinical use. Meanwhile, the polydispersity index of particle size distribution was decreased from 0.106 to 0.06, which made the micellar particle size distribution more uniform and facilitated the exertion of drug efficacy. The micelle prepared by spray drying remained stable for 6 hours at room temperature and 24 hours at 4° C. Compared with the film hydration method, the stability of the micelle was greatly improved.

### Release Test Conditions (Nanocage Reciprocating Rack Method):

Dialysis bag: regenerated cellulose dialysis bag (8000-10000)
Solution in dialysis bag: aqueous solution containing approximately 0.2 mg/ml of Cabazitaxel
Medium name: pH 7.4 phosphate buffer (containing 0.1% Tween 80)
Medium volume: 50 ml
Sampling volume: 5 ml
Sampling time points: 0.5 h, 2 h, 4 h, 8 h, 12 h, 16 h, 20 h, 24 h, 36 h, 48 h.

### Zeta Potential Test Method:

Took one bottle of the test sample (taken out and placed at room temperature), shook the bottle wall to ensure that the lyophilized powder was completely detached from the bottle wall. Used a syringe to inject 20 ml of 5% glucose injection (ensuring that all the powder was wetted during the injection process), shook it slowly several times, let it stand for 5 minutes, and released the gas in the bottle with the syringe needle. Let it stand until completely dissolved (could shake it slowly a few times during the process), shook it well, and prepared a solution containing 3 mg of Cabazitaxel per 1 mL as the test sample solution for potential. Measured its potential according to the following instrument method.
Determination temperature: 25° C.
Medium: Water
Resolvent: 5% glucose injection
Sample concentration (mg/mL): 3 mg/mL
(Note: (Water) Refractive index: 1.33. Viscosity: 0.8872. Dielectric constant: 78.5)

### Example 2

Following the method of Example 1, three batches of Cabazitaxel micellar preparations with Cabazitaxel: DSPE-PEG2000 (CAS: 147867-65-0) mass ratios of 1:7 (batch number 3662107271), 1:11 (batch number 3662107241), and 1:15 (batch number 3662107272) were respectively prepared (the mass ratio of Cabazitaxel:DSPE-PEG2000 in the pre-spray drying solution was 1:1, and the remaining DSPE-PEG2000 was added during the hydration of the spray-dried powder). The results of screening the API/DSPE-PEG2000 ratio for formulation quality were shown in Table 2, and the appearance of the hydrated drug solution was shown in FIGS. 3-5.

TABLE 2
Results of Formulation Excipient Ratio Screening

| API:Cultured | | | | | | |
|---|---|---|---|---|---|---|
| Phosphatidylethanolamine(mass | | | | | | |
| ratio) | | 1:7 | 1:11 | | 1:15 | |
| Properties (After hydration, | | White turbid | Clear transparent | | Clear transparent | |
| unfiltered, unfrozen-dried) | | solution | solution | | solution | |
| Properties (After reconstitution | | / | Clear transparent | | Clear transparent | |
| from freeze-drying) | | | solution | | solution | |
| Reconstitution time | | / | 10 | min | 45 | min |
| Encapsulation rate (%) | | 98.00 | 99.70 | | 98.90 | |
| Related substances (%) | | Impurity A: 0.01. | Impurity A: 0.01. | | Impurity A: 0.01. | |
| | | Impurity I: 0.02. | Impurity I: 0.01. | | Impurity I: 0.02. | |
| | | Impurity J: 0.03. | Impurity J: 0.04. | | Impurity J: 0.03. | |
| | | Total impurities: 0.06 | Total impurities: 0.06 | | Total impurities: 0.06 | |
| Particle size | Particle size | Solution is turbid, | 15 | nm | 13 | nm |
| and particle | (nm) | particle size cannot | | | | |
| size | | be detected | | | | |
| distribution | Polydispersity | / | 0.06 | | 0.04 | |
| | index | | | | | |

Note:
“/” indicates not freeze-dried

The above results showed that when the ratio of API to phosphatidylethanolamine was 1:7, the hydration solution was turbid, indicating that phosphatidylethanolamine could not encapsulate the corresponding amount of Cabazitaxel active substance, and the encapsulation rate was 98.0%, which was significantly lower than those of the micellar samples with API to excipient ratios of 1:11 and 1:15. When the API to excipient ratios were 1:11 and 1:15, the hydration solutions were both clear and transparent; the related substances, particle size and particle size distribution of the samples were comparable, and the micellar preparations were uniform. However, this formulation was an injection; according to the principle of minimum excipient input, the ratio of API to phosphatidylethanolamine of 1:11 was selected as the optimal ratio for subsequent sample preparation to reduce the production cost of the formulation.

### Example 3

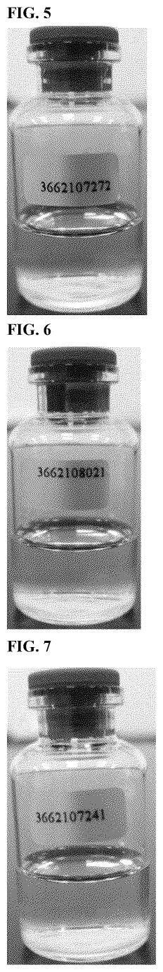

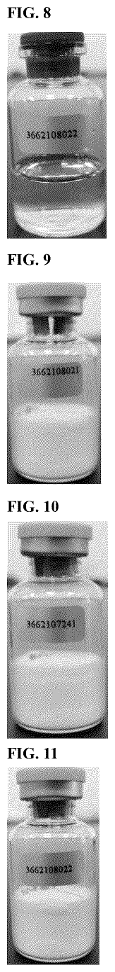

Referring to Example 2, the hydration solution of Cabazitaxel and PEG2000-DSPE was prepared at an API to excipient ratio of 1:11. Cabazitaxel micellar formulations without freeze-drying protectants, with 4% sucrose (mass/volume ratio of the freeze-drying protectant to the pre-freeze-drying micellar solution, w/v), and with 4% lactose were respectively prepared. The specific results were shown in Table 3. The appearance diagrams of the finished products after freeze-drying with different types of freeze-drying protectants as well as the reconstitution solutions of the finished products were respectively shown in FIGS. 6 to 11.

TABLE 3
Results of Lyoprotectant Screening

| Types of lyoprotectant | | / | 4% Sucrose | 4% Lactose |
|---|---|---|---|---|
| Batch number of samples | | 3662107241 | 3662108021 | 3662108022 |
| Properties of the reconstituted | | Clear transparent | Clear transparent | Clear transparent |
| solution | | solution | solution | solution |
| Reconstitution time | | 10 min | 3 min | 6 min |
| Encapsulation efficiency (%) | | 99.7 | 99.7 | 99.9 |
| Particle size | Particle size | 15 | 15 | 16 |
| and particle | (nm) | | | |
| size | Polydispersity | 0.06 | 0.1 | 0.07 |
| distribution | index | | | |

| Related substances (%) | Impurity A: 0.01. | Impurity A: 0.01. | Impurity A: 0.01. |
|---|---|---|---|
| | Impurity I: 0.01. | Impurity I: 0.01. | Impurity I: 0.02. |
| | Impurity J: 0.04. | Impurity J: 0.03. | Impurity J: 0.04. |
| | Total impurities: 0.06 | Total impurities: 0.05 | Others: 0.11. |
| | | | Total impurities: 0.42 |

Note:
“/” represents “none”.

From the above results, the properties, entrapment efficiency, particle size and particle size distribution of the three batches of preparations after reconstitution were not significantly different. However, the total impurity content of the micellar preparation made with 4% lactose as the freeze-drying protectant was 0.42%, which was significantly higher than that of the micellar preparations without freeze-drying protectant and with 4% sucrose. To reduce production costs and follow the principle of using the minimum amount of excipients, no freeze-drying protectant was added in the subsequent preparations to reduce the use of excipients.

### Example 4

Three batches of Cabazitaxel micellar reconstitution solutions were prepared according to the preparation method of Example 1. The reconstitution solutions were filtered twice through a 0.22 μm PVDF membrane to obtain the filtrate. The composition of the small-scale confirmation batch of the reconstitution solution was shown in Table 4, and the test results were shown in Table 5. The above filtration was filled into medium borosilicate glass vials and freeze-dried to obtain the preparation samples.

TABLE 4
Composition of Pilot-Scale Confirmation Batch

| | |
|---|---|
| Batch number | 3662108191/3662108192/3662108193 |
| Material Name | Single-dose formulation (mg) |
| Cabazitaxel | 60 |
| Pegylated | 660 |
| phosphatidylethanolamine | |
| Anhydrous Ethanol ^{[1]} | 1 ml |
| Purified Water | 9.28 |

Note ^{[1]}:
Anhydrous ethanol is used as a solvent and is removed during the spray drying process.

TABLE 5
Test Results of Pilot-Scale Confirmation Batch

| Batch number | | 3662108191 | 3662108192 | 3662108193 |
|---|---|---|---|---|
| Appearance | | White loose solid | White loose solid | White loose solid |
| Reconstitution time | | 10 min | 5 min | 7 min |
| Particle size | Particle size | 15 | 14 | 15 |
| and particle | (nm) | | | |
| size | Polydispersity | 0.04 | 0.04 | 0.06 |
| distribution | index (PDI) | | | |
| pH | | 5.8 | 5.8 | 5.8 |
| Encapsulation rate (%) | | 99.8 | 99.0 | 96.6 |
| Content (%) | | 103.3 | 99.5 | 103.4 |
| Related | Impurity A | 0.01 | 0.01 | 0.01 |
| substances | Impurity I | 0.03 | 0.02 | 0.02 |
| (%) | Impurity J | 0.04 | 0.03 | 0.04 |
| | Total | 0.08 | 0.06 | 0.07 |
| | impurities | | | |

The results showed that the three batches of finished products prepared by the proposed formula and process in the pilot test met the quality standards, and there was no significant difference between the batches, indicating that the proposed formula and process had good reproducibility and could be further scaled up for production.

The samples obtained from the confirmed pilot test batches were placed at 25° C.±2° C./60% RH±10% RH for 2 weeks, 4 weeks, and 8 weeks; and at 40° C.±2° C./75% RH±5% RH for 1 week, 2 weeks, and 4 weeks to investigate the stability of the samples under each condition. The results were shown in Tables 6 to 8.

TABLE 6
Test Results of the Pilot -Scale Confirmation Batch (Batch No.: 3662108191)

| / | | 40 °C ± 2 °C/75% RH ± 5% RH | | | | 25 °C ± 2 °C/60% RH ± 10% RH | | | |
|---|---|---|---|---|---|---|---|---|---|
| Test items | | 0 [symbol] | 1 Week | 2 Week | 4 Week | 0 day | 2 Week | 4 Week | 8 Week |
| Content % | | 103.3 | 104.5 | 101.8 | 101.6 | 103.3 | 102.5 | 102.8 | 99.1 |
| Encapsulation rate % | | 99.8 | 99.2 | 100.3 | 99.6 | 99.8 | 99.3 | 99.9 | 100.0 |
| pH | | 5.8 | 5.8 | 5.5 | 5.6 | 5.8 | 5.6 | 5.7 | 5.7 |
| Related | Impurity A % | 0.01 | 0.01 | 0.01 | 0.01 | 0.01 | 0.01 | 0.01 | 0.01 |
| substances | Impurity B % | Not | Not | Not | Not | Not | Not | Not | Not |
| (%) | | detected | detected | detected | detected | detected | detected | detected | detected |
| | Impurity C % | Not | Not | Not | Not | Not | Not | Not | Not |
| | | detected | detected | detected | detected | detected | detected | detected | detected |
| | Impurity D % | Not | Not | Not | Not | Not | Not | Not | Not |
| | | detected | detected | detected | detected | detected | detected | detected | detected |
| | Impurity G % | Not | Not | Not | Not | Not | Not | Not | Not |
| | | detected | detected | detected | detected | detected | detected | detected | detected |
| | Impurity H % | Not | Not | Not | Not | Not | Not | Not | Not |
| | | detected | detected | detected | detected | detected | detected | detected | detected |
| | Impurity I % | 0.03 | 0.02 | 0.01 | 0.01 | 0.03 | 0.02 | 0.01 | 0.01 |
| | Impurity J % | 0.04 | 0.04 | 0.04 | 0.04 | 0.04 | 0.04 | 0.04 | 0.03 |
| | Other | <0.05 | <0.05 | <0.05 | <0.05 | <0.05 | <0.05 | <0.05 | <0.05 |
| | Impurity % | | | | | | | | |
| | Total | 0.08 | 0.07 | 0.06 | 0.06 | 0.08 | 0.07 | 0.06 | 0.05 |
| | impurities % | | | | | | | | |

TABLE 7
Test Results of the Pilot-Scale Confirmation Batch (Batch No.: 3662108192)

| / | | 40 °C ± 2 °C/75% RH ± 5% RH | | | | 25 °C ± 2 °C/60% RH ± 10% RH | | | |
|---|---|---|---|---|---|---|---|---|---|
| Test items | | 0 [symbol] | 1 Week | 2 Week | 4 Week | 0 day | 2 Week | 4 Week | 8 Week |
| Content % | | 99.5 | 101.4 | 101.8 | 99.9 | 99.5 | 99.1 | 98.2 | 102.2 |
| Encapsulation rate % | | 99.0 | 99.6 | 99.3 | 98.3 | 99.0 | 99.0 | 99.4 | 100.4 |
| pH | | 5.8 | 5.8 | 5.5 | 5.8 | 5.8 | 5.6 | 5.8 | 5.7 |
| Related | Impurity A % | 0.01 | 0.01 | 0.01 | 0.01 | 0.01 | 0.01 | 0.01 | 0.01 |
| substances | Impurity B % | Not | Not | Not | Not | Not | Not | Not | Not |
| (%) | | detected | detected | detected | detected | detected | detected | detected | detected |
| | Impurity C % | Not | Not | Not | Not | Not | Not | Not | Not |
| | | detected | detected | detected | detected | detected | detected | detected | detected |
| | Impurity D % | Not | Not | Not | Not | Not | Not | Not | Not |
| | | detected | detected | detected | detected | detected | detected | detected | detected |
| | Impurity G % | Not | Not | Not | Not | Not | Not | Not | Not |
| | | detected | detected | detected | detected | detected | detected | detected | detected |
| | Impurity H % | Not | Not | Not | Not | Not | Not | Not | Not |
| | | detected | detected | detected | detected | detected | detected | detected | detected |
| | Impurity I % | 0.02 | 0.02 | 0.01 | 0.01 | 0.02 | 0.01 | 0.01 | 0.01 |
| | Impurity J % | 0.03 | 0.04 | 0.04 | 0.04 | 0.03 | 0.04 | 0.04 | 0.04 |
| | Other | <0.05 | <0.05 | 0.29 | <0.05 | <0.05 | 0.36 | <0.05 | <0.05 |
| | Impurity % | | | | | | | | |
| | Total | 0.06 | 0.07 | 1.27 | 0.06 | 0.06 | 1.89 | 0.06 | 0.06 |
| | impurities % | | | | | | | | |

TABLE 8
Test Results of the Pilot-Scale Confirmation Batch (Batch No.: 3662108193)

| / | | 40 °C ± 2 °C/75% RH ± 5% RH | | | | 25 °C ± 2 °C/60% RH ± 10% RH | | | |
|---|---|---|---|---|---|---|---|---|---|
| Test items | | 0 [symbol] | 1 Week | 2 Week | 4 Week | 0 day | 2 Week | 4 Week | 8 Week |
| Content % | | 103.4 | 105.0 | 101.4 | 102.2 | 103.4 | 91.1 | 102.3 | 95.8 |
| Encapsulation rate % | | 96.6 | 97.4 | 99.6 | 98.5 | 96.6 | 102.4 | 95.8 | 97.1 |
| pH | | 5.8 | 5.8 | 5.6 | 5.6 | 5.8 | 5.6 | 5.6 | 5.7 |
| Related | Impurity A % | 0.01 | 0.01 | 0.01 | 0.01 | 0.01 | 0.01 | 0.01 | 0.01 |
| substances | Impurity B % | Not | Not | Not | Not | Not | Not | Not | Not |
| (%) | | detected | detected | detected | detected | detected | detected | detected | detected |
| | Impurity C % | Not | Not | Not | Not | Not | Not | Not | Not |
| | | detected | detected | detected | detected | detected | detected | detected | detected |
| | Impurity D % | Not | Not | Not | Not | Not | Not | Not | Not |
| | | detected | detected | detected | detected | detected | detected | detected | detected |
| | Impurity G % | Not | Not | Not | Not | Not | Not | Not | Not |
| | | detected | detected | detected | detected | detected | detected | detected | detected |
| | Impurity H % | Not | Not | Not | Not | Not | Not | Not | Not |
| | | detected | detected | detected | detected | detected | detected | detected | detected |
| | Impurity I % | 0.02 | 0.03 | 0.01 | 0.01 | 0.02 | 0.01 | 0.01 | 0.01 |
| | Impurity J % | 0.04 | 0.04 | 0.04 | 0.04 | 0.04 | 0.04 | 0.04 | 0.04 |
| | Other | <0.05 | <0.05 | <0.05 | 0.05 | <0.05 | <0.05 | <0.05 | <0.05 |
| | Impurity % | | | | | | | | |
| | Total | 0.07 | 0.08 | 0.06 | 0.11 | 0.07 | 0.11 | 0.06 | 0.06 |
| | impurities % | | | | | | | | |

The above results indicated that the three batches of preparations prepared by the pilot-scale process, when stored at 40° C.±2° C./75% RH±5% RH for 4 weeks and at 25° C.±2° C./60% RH±10% RH for 8 weeks, showed no significant changes in content, entrapment efficiency, pH value, and related substances compared to the initial point, and all met the proposed quality standards.

This suggests that the pilot-scale process was stable and could be used for further scale-up production.

### Example 5: Pharmacokinetic Study of Cabazitaxel

Preparation of the administered formulation: Took one bottle of the test sample (according to the specification), gently shook the bottle to disperse the contents into powder. Then, 20 mL of 5% glucose solution was drawn using a sterile syringe. The glucose solution was automatically injected along the inner wall of the vial under negative pressure. The bottle was gently shaken to ensure complete dispersion and dissolution, to prepare a formulation with a concentration of 3 mg/mL. A total of six beagle dogs, with equal numbers of males and females, were used in this experiment. Each beagle dog was intravenously infused with a single dose of 0.5 mg/kg of the spray-dried and rehydrated Cabazitaxel micelle prepared as described in Example 4 over 15 minutes. Blood samples were collected from all test animals via venipuncture at predetermined time points before and after administration, and plasma was separated. The average concentrations of free and total Cabazitaxel in dog plasma were determined by conventional analytical methods in the art.

TABLE 9
Summary of the average concentrations of free and total Cabazitaxel in the plasma
of beagle dogs after a single intravenous infusion of Cabazitaxel micelles (ng/mL)

| | Cabazitaxel Concentration (ng/mL) | | | | | | | |
|---|---|---|---|---|---|---|---|---|
| Time | Free | | | | Total | | | |
| (h) | N | Mean | SD | CV % | N | Mean | SD | CV % |
| 0 | 6 | 0.00 | 0.00 | NA | 6 | 0.00 | 0.00 | NA |
| 0.083 | 6 | <LOQ | <LOQ | 0 | 6 | 75.0 | 36.0 | 48.1 |
| 0.25 | 6 | <LOQ | <LOQ | NA | 6 | 122 | 19.6 | 16.1 |
| 0.33 | 6 | <LOQ | <LOQ | NA | 6 | 20.3 | 1.58 | 7.8 |
| 0.5 | 6 | <LOQ | <LOQ | NA | 6 | 8.38 | 1.17 | 13.9 |
| 0.75 | 6 | <LOQ | <LOQ | NA | 6 | 5.96 | 0.735 | 12.3 |
| 1 | 6 | <LOQ | <LOQ | NA | 6 | 5.50 | 0.496 | 9.0 |
| 2 | 6 | <LOQ | <LOQ | NA | 6 | 3.77 | 0.720 | 19.1 |
| 4 | 6 | <LOQ | <LOQ | NA | 6 | 3.22 | 0.645 | 20.0 |
| 8 | 6 | <LOQ | <LOQ | NA | 6 | 2.11 | 0.514 | 24.3 |
| 24 | 6 | <LOQ | <LOQ | NA | 6 | 1.38 | 0.279 | 20.3 |

Note:
Mean represents the average value, SD represents the standard deviation, CV % represents the coefficient of variation, NA indicates not applicable, and <LOQ indicates below the limit of quantification.

After Cabazitaxel micelles enter the bloodstream, the drug remains encapsulated in the micelles during circulation. The drug-loaded micelles directly and rapidly enter the lesion tissue cells to exert their therapeutic effect.

### Example 6: Efficacy of Cabazitaxel Micelles

Preparation method of positive control Cabazitaxel solution: Took Cabazitaxel injection (Manufacturer: Sanofi-Aventis U.S. LLC.; Specification: 60 mg: 1.5 ml; the injection contains Cabazitaxel, ethanol, and polysorbate 80) and placed it at room temperature for 5 minutes. Used a syringe to draw out the Cabazitaxel injection and injected it into another Cabazitaxel injection vial. Diluted the Cabazitaxel injection with the solvent provided in the injection to a concentration of 10 mg/mL. Inverted the vial repeatedly to mix for at least 45 seconds, without shaking. Let the vial stand at room temperature for 5 minutes until completely dissolved. Then diluted with 5% glucose solution to concentrations of 1.5 mg/mL and 0.75 mg/mL. Used within 4 hours under room temperature and normal light conditions.

Preparation method of test Cabazitaxel micelles: Under sterile conditions, took the formulation sample obtained by freeze-drying in Example 1 and stored in a borosilicate glass vial as the test sample. Each vial of the test sample was dispersed and dissolved in a certain volume of 5% glucose solution. Shook the vial to completely disperse and dissolve the freeze-dried powder. A certain amount of foam was be produced under negative pressure in the vial; injected air to balance the pressure, and the foam subsided. After preparation, the drug solution was placed at room temperature for 30 minutes to ensure complete dissolution. Diluted with 5% glucose solution to concentrations of 3 mg/mL, 1.5 mg/mL, and 0.75 mg/mL.

When the tumor grew to 101.05-167.11 mm³, 36 NPG mice (NOD-Prkdcscid Il2rgnull mice; Grade: SPF; Source: Beijing Vitora Biotechnology Co., Ltd.) were randomly divided into 6 groups, with 6 mice in each group, namely the solvent control group (5% glucose solution), positive control Cabazitaxel solution groups (7.5, 15 mg/kg), and test Cabazitaxel micelle groups (7.5, 15, 30 mg/kg). Intravenous injection was administered once a week for 4 consecutive weeks.

Throughout the experiment, in the solvent control group, the tumors grew gradually; at the end of the experiment (Day 29, abbreviated as D29, the same abbreviation is used below), the average tumor volume was 1749.33±673.47 mm³, and the average RTV (Relative Tumor Volume) was 13.19±4.78. In the positive control Cabazitaxel solution groups (7.5, 15 mg/kg), the average tumor volumes were 612.97±234.46 mm³ and 313.74±74.53 mm³, respectively, and the average RTVs were 4.74±1.79 and 2.29±0.39, respectively. From D19 to D29, the average tumor volume and RTV of the 15 mg/kg positive control group were significantly lower than those of the solvent control group (p<0.05). At some time points, the average tumor volume and RTV of the 7.5 mg/kg positive control group were significantly lower than those of the solvent control group (p<0.05); at other time points, the average tumor volume and RTV of the 7.5 mg/kg and 15 mg/kg positive control groups were lower than those of the solvent control group, but there was no statistical difference (p>0.05). In the Cabazitaxel micelle groups (7.5, 15, 30 mg/kg), the average tumor volumes were 209.14±34.20 mm³, 114.96±51.91 mm³, and 99.02±37.56 mm³, respectively; and the average relative tumor volumes (RTV) were 1.57±0.24, 0.86±0.37, and 0.72±0.23, respectively. On D8 and from D15 to D29, the average tumor volumes of the 7.5, 15, and 30 mg/kg Cabazitaxel micelle groups were significantly lower than those of the solvent control group, and from D12 to D29, their RTVs were significantly lower (p≤0.05). From D26 to D29, the average tumor volume and RTV of the 30 mg/kg group were significantly lower than those of the 7.5 mg/kg Cabazitaxel micelle group (p≤0.05). At a dose of 15 mg/kg, from D19 to D29, the average tumor volume of the test sample, and from D15 to D29, its RTV were significantly lower than those of the positive control group at the same dose (p≤0.05).

At the end of the experiment (D29), the relative tumor proliferation rates (T/C %) of the positive control Cabazitaxel solution groups (7.5, 15 mg/kg) and the Cabazitaxel micelle groups (7.5, 15, 30 mg/kg) were 35.90%, 17.38%, 11.92%, 6.49%, and 5.49%, respectively. Although the T/C % value of the 7.5 mg/kg positive control group decreased below 40%, there was no statistical difference in RTV compared with the solvent control group. In the middle and later stages of the experiment, the T/C % values of the other groups decreased below 40%. On D29, at the time of euthanasia, the relative tumor volume inhibition rates (IRTV %) of the positive control Cabazitaxel solution groups (7.5, 15 mg/kg) and the Cabazitaxel micelle groups (7.5, 15, 30 mg/kg) were 64.10%, 82.62%, 88.08%, 93.51%, and 94.51%, respectively.

At the end of the experiment, after euthanasia, the tumor weights were measured. The average tumor weights of the solevent control group, positive control Cabazitaxel solution groups (7.5, 15 mg/kg), and Cabazitaxel micelle groups (7.5, 15, 30 mg/kg) were 0.853±0.278 g, 0.256±0.107 g, 0.127±0.034 g, 0.086±0.020 g, 0.049±0.031 g, and 0.032±0.013 g, respectively. The tumor weights of the positive control and test sample groups were significantly lower than those of the solevent control group (p≤0.05). The tumor weight of the 15 mg/kg test sample group was significantly lower than that of the positive control group at the same dose (p≤0.05), and the tumor weight of the 30 mg/kg test sample group was significantly lower than that of the 7.5 mg/kg test sample group (p≤0.05). There was no statistical difference in tumor weight between the 7.5 mg/kg test sample group and the positive control group at the same dose (p>0.05). The relative tumor weight inhibition rates (IRTW %) of the solevent control, positive control, and test sample groups were 69.95%, 85.09%, 89.94%, 94.22%, and 96.21%, respectively.

Compared with the 7.5 mg/kg positive control Cabazitaxel solution, the 7.5 mg/kg Cabazitaxel micelle had significant advantages and statistical differences in TV (tumor volume) and RTV from D15 to D29; the tumor weight of the 7.5 mg/kg Cabazitaxel micelle also had significant advantages and statistical differences.

Compared with the 15 mg/kg positive control Cabazitaxel solution, the 15 mg/kg Cabazitaxel micelle had significant advantages and statistical differences in TV and RTV from D15 to D29; the tumor weight of the 15 mg/kg Cabazitaxel micelle also had significant advantages and statistical differences.

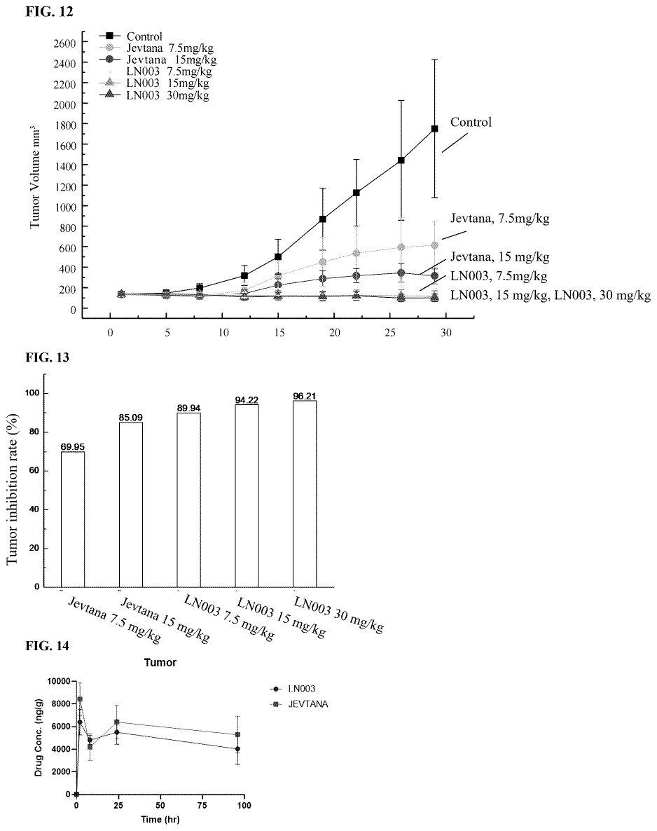

Compared with the 15 mg/kg positive control Cabazitaxel solution, the 7.5 mg/kg Cabazitaxel micelle showed significant advantages and statistical differences in TV and RTV from D15 to D29. The above results indicated that at the same dose, both the 7.5 mg/kg and 15 mg/kg Cabazitaxel micelles (test samples) had significant efficacy advantages over the positive control Cabazitaxel solution (positive control sample). Furthermore, the 7.5 mg/kg Cabazitaxel micelle also had significant efficacy advantages over the 15 mg/kg positive control Cabazitaxel solution. The tumor growth trend graphs of each group were shown in FIG. 12, and the tumor weight inhibition rate graphs were shown in FIG. 13.

### Example 7: Cabazitaxel Micelles Reduce Drug Distribution in the Brain

Preparation of the test Cabazitaxel micelles: Took the formulation sample obtained by freeze-drying in Example 1 and stored in aa injection-grade borosilicate glass vial as the test sample. Took one vial of the test sample (according to the specification), gently shook the vial to disperse the powder, then used a sterile syringe to draw 20 mL of 5% glucose solution. The glucose solution was automatically injected along the inner wall of the vial under negative pressure. The vial was gently shaken to achieve complete dispersion and dissolution, and the resulting solution with a Cabazitaxel concentration of 3 mg/mL, was as used as the administration formulation.

Preparation of the reference JEVTANA solution: Took one vial of JEVTANA, slowly added all of the solvent along the inner wall of the vial, then gently inverted the vial up and down repeatedly for at least 45 seconds. No visible particles were observed, and the resulting solution with a concentration of 10 mg/mL was used as the stock solution. Took an appropriate amount of the stock solution into a sterile container and diluted it with 5% glucose injection to the required concentration.

Forty-eight male Balb/c nude tumor-bearing mice were divided into two groups (24 mice per group), with 4 sampling points per group. Balb/c nude tumor-bearing mice (SPF grade. source: Shanghai Lingchang Biotechnology Co., Ltd.) were given a single intravenous injection of 30 mg/kg of the test Cabazitaxel micelle or 30 mg/kg of the reference JEVTANA solution. Blood and tissue samples were collected at 2 h, 8 h, 24 h, and 96 h after administration to investigate the tissue distribution characteristics of Cabazitaxel in Balb/c nude tumor-bearing mice.

### Test Methods:

### Blood Sample Collection

Animals for blood collection: All animals
Blood collection site: Blood was collected from the heart of the animals after euthanasia at the sampling points.
Blood collection time points: 2 h, 8 h, 24 h, and 96 h after administration (sample number: group number—plasma).
Blood collection time error: ±5% after administration.
Blood collection volume: All blood samples/per time point Blood collection tube: Heparin sodium anticoagulant
Sample processing and storage: After collected into blood collection tubes, the sample were shaken up and down 5 to 8 times for thorough mixing, placed on wet ice prior to centrifugation, and centrifuged at 4000 rpm for 5 minutes at 4° C. within 1 hour to separate plasma. The plasma was divided into two moieties (one moiety for testing, the other for backup) and transferred into labeled EP tubes. The samples were transferred into an ultra-low temperature at refrigerator at −70±10° C. within 30 minutes and stored until shipment.

### Tissue Collection

Animals for tissue collection: All animals
Tissue collection site: Tissues were collected after euthanasia of the animals.
Tissue collection time points: 2 h, 8 h, 24 h, and 96 h after administration (sample number: group number—tissue name).
Tissue types: Tumor, heart, liver, spleen, lung, kidney, brain, stomach, lymph node, duodenum, testis (reproductive gland)
Collection volume: The entire tissue
Tissue collection tube: Ordinary sample collection tube
Sample processing and storage: After the tumor was collected, heart perfusion was performed with ice-cold 0.9% sodium chloride injection, followed by collection of the required tissue samples. After collection, the samples were rinsed to remove residual blood and other secretions, blotted dry with filter paper, weighed, and placed in labeled EP tubes. The samples were temporarily stored on dry ice. After sample collection at each time point, all samples were stored in an ultra-low temperature refrigerator at −70±10° C. until shipment.

### Pharmacokinetic Bioanalysis

The concentration of Cabazitaxel in all animal plasma and tissue samples was detected by LC-MS/MS.

### Data Processing and Statistics

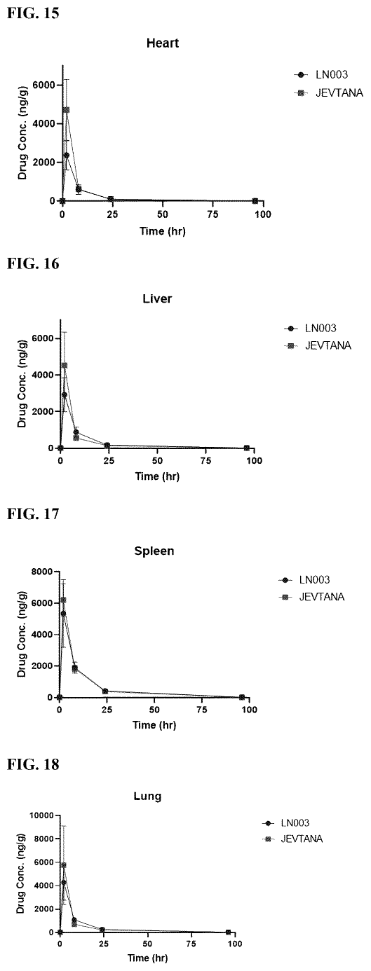

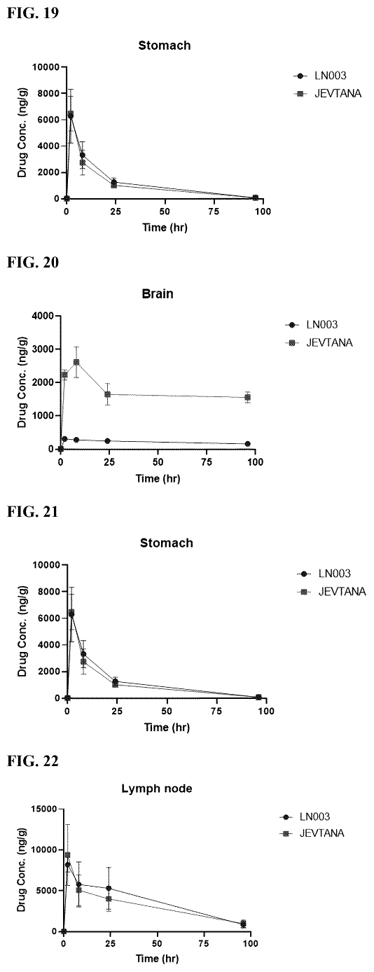

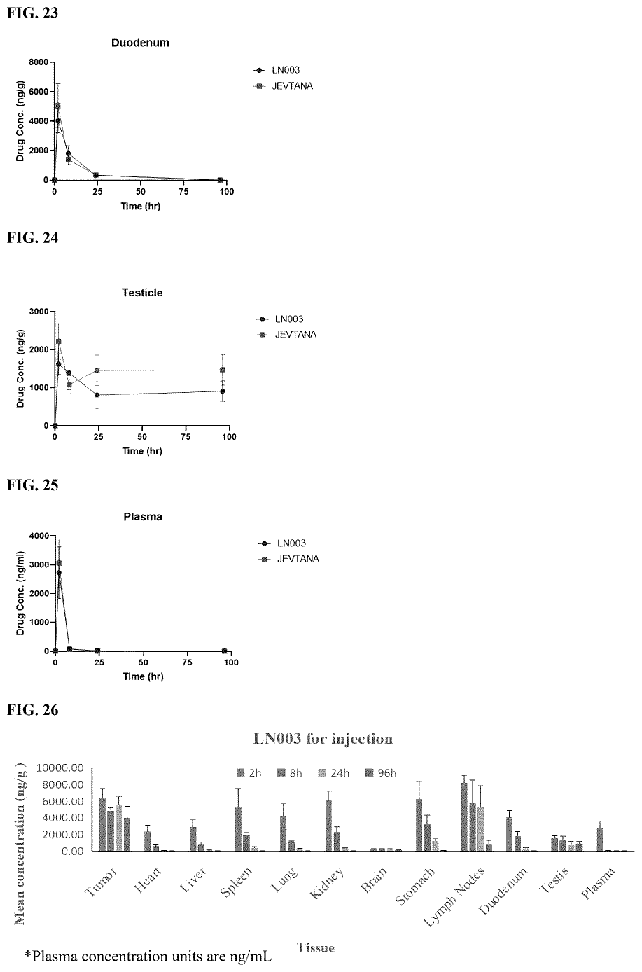

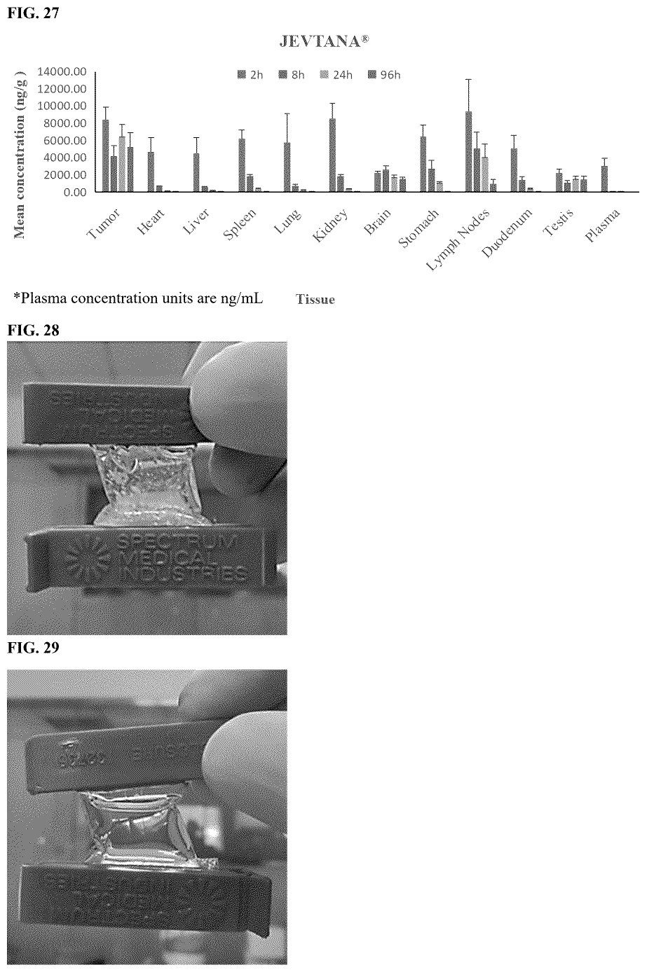

Pharmacokinetic parameters were calculated using Phoenix WinNonlin 8.3.3. Pharmacokinetic parameters were analyzed and summarized using either Phoenix WinNonlin 8.3.3 software or Excel 2013. After intravenous injection of 30 mg/kg of the test injectable Cabazitaxel micelle or JEVTANA, the mean drug concentrations in the tissues and plasma of tumor-bearing mice reached their peaks at 2 h. Among all tissues, the drug concentration in the lymph nodes was the highest, with peak concentrations of 8186.67 ng/g and 9380.00 ng/g, respectively. The peak plasma drug concentrations were 2723.33±898.90 ng/ml and 3050.00±849.99 ng/mL, respectively. The peak drug concentrations in the kidney tissues were 6211.67±983.06 ng/g and 6466.67±1328.78 ng/g, respectively. The drug concentrations in the tumor tissues of both groups reached their peaks at 2 h, with values of 6391.67 ng/g and 8433.33 ng/g, respectively. The distribution of the drug in the brain tissue was as follows: the test injectable Cabazitaxel micelle (Group 1) reached a peak of 303.92 ng/g at 2 h and then showed a certain decrease over time, while JEVTANA (Group 2) reached a peak of 2612.50 ng/g at 8 h. The peak drug concentration in the brain of Group 1 was 11.6% of that of Group 2. The average drug concentrations are summarized in Tables 10-11. The average drug-time curves of Cabazitaxel in each tissue and plasma are shown in FIGS. 14-25, and the average drug concentration bar charts are shown in FIGS. 26-27 (the unit of tissue concentration is ng/g, and the unit of plasma concentration is ng/ml. in FIGS. 26 and 27, each group of data involves the average drug concentrations at four time points, from left to right, the data columns represent the average drug concentrations at 2 h, 8 h, 24 h, and 96 h, respectively). “Injectable LN003” in FIGS. 14-25 has the same meaning as the “test Cabazitaxel micelle” described in Example 7.

TABLE 10
Average drug concentration of Cabazitaxel in tissues and plasma of tumor-bearing
mice after injection of the test Cabazitaxel micelles for injection

| | | | | | | | | | | Lymph | | | |
|---|---|---|---|---|---|---|---|---|---|---|---|---|---|
| Time | | Tumor | Heart | Liver | Spleen | Lung | Kidney | Brain | Stomach | node | Duodenum | Testis | Plasma |
| 2 h | N | 6 | 6 | 6 | 6 | 6 | 6 | 6 | 6 | 6 | 6 | 6 | 6 |
| | Mean | 6391.67 | 2370.83 | 2914.17 | 5343.33 | 4267.50 | 6211.67 | 303.92 | 6282.50 | 8186.67 | 4035.83 | 1620.83 | 2723.33 |
| | SD | 1126.68 | 758.87 | 919.99 | 2159.52 | 1512.55 | 983.06 | 20.36 | 2053.81 | 908.77 | 820.55 | 274.60 | 898.90 |
| | CV % | 17.6 | 32.0 | 31.6 | 40.4 | 35.4 | 15.8 | 6.7 | 32.7 | 11.1 | 20.3 | 16.9 | 33.0 |
| 8 h | N | 6 | 6 | 6 | 6 | 6 | 6 | 6 | 6 | 6 | 6 | 6 | 6 |
| | Mean | 4810.00 | 595.25 | 871.67 | 1903.33 | 1075.83 | 2299.17 | 277.42 | 3315.00 | 5770.00 | 1822.50 | 1390.00 | 78.28 |
| | SD | 402.23 | 253.39 | 266.21 | 356.28 | 151.31 | 619.94 | 23.02 | 1021.15 | 2757.93 | 521.79 | 443.75 | 30.20 |
| | CV % | 8.4 | 42.6 | 30.5 | 18.7 | 14.1 | 27.0 | 8.3 | 30.8 | 47.8 | 28.6 | 31.9 | 38.6 |
| 24 h | N | 6 | 6 | 6 | 6 | 6 | 6 | 6 | 6 | 6 | 6 | 6 | 6 |
| | Mean | 5507.50 | 100.83 | 169.67 | 418.17 | 251.67 | 354.50 | 243.50 | 1254.17 | 5296.67 | 325.83 | 809.25 | 13.82 |
| | SD | 1071.54 | 25.87 | 27.60 | 117.20 | 83.55 | 87.74 | 42.45 | 319.68 | 2547.67 | 119.11 | 347.61 | 1.59 |
| | CV % | 19.5 | 25.7 | 16.3 | 28.0 | 33.2 | 24.8 | 17.4 | 25.5 | 48.1 | 36.6 | 43.0 | 11.5 |
| 96 h | N | 6 | 3 | 5 | 5 | 5 | 6 | 6 | 6 | 6 | 6 | 6 | 4 |
| | Mean | 4028.33 | 7.65 | 7.14 | 7.59 | 16.57 | 14.98 | 158.42 | 64.27 | 847.67 | 7.53 | 907.92 | 1.85 |
| | SD | 1365.94 | 3.47 | 1.56 | 1.46 | 4.71 | 1.33 | 12.66 | 21.65 | 426.41 | 1.81 | 268.23 | 0.35 |
| | CV % | 33.9 | 45.3 | 21.8 | 19.2 | 28.4 | 8.9 | 8.0 | 33.7 | 50.3 | 24.1 | 29.5 | 19.0 |

Note:
The unit of tissue concentration is (ng/g), and the unit of plasma concentration is (ng/mL).

TABLE 11
Average drug concentrations of Cabazitaxel in tissues and plasma of tumor-bearing mice after injection of JEVTANA for injection

| | | | | | | | | | | Lymph | | | |
|---|---|---|---|---|---|---|---|---|---|---|---|---|---|
| Time | | Tumor | Heart | Liver | Spleen | Lung | Kidney | Brain | Stomach | node | Duodenum | Testis | Plasma |
| 2 h | N | 6 | 6 | 6 | 6 | 6 | 6 | 6 | 6 | 6 | 6 | 6 | 6 |
| | Mean | 8433.33 | 4709.17 | 4520.00 | 6204.17 | 5745.00 | 8566.67 | 2230.00 | 6466.67 | 9380.00 | 5070.00 | 2219.17 | 3050.00 |
| | SD | 1446.61 | 1590.70 | 1822.36 | 1029.37 | 3357.16 | 1772.76 | 155.56 | 1328.78 | 3741.19 | 1500.78 | 471.37 | 849.99 |
| | CV % | 17.2 | 33.8 | 40.3 | 16.6 | 58.4 | 20.7 | 7.0 | 20.5 | 39.9 | 29.6 | 21.2 | 27.9 |
| 8 h | N | 6 | 6 | 6 | 6 | 6 | 6 | 6 | 6 | 6 | 6 | 6 | 6 |
| | Mean | 4199.17 | 622.50 | 559.58 | 1838.33 | 689.42 | 1855.83 | 2612.50 | 2740.00 | 5056.67 | 1415.83 | 1078.33 | 68.98 |
| | SD | 1192.46 | 90.15 | 63.02 | 192.22 | 206.40 | 150.31 | 464.14 | 949.18 | 1893.33 | 368.50 | 238.24 | 26.21 |
| | CV % | 28.4 | 14.5 | 11.3 | 10.5 | 29.9 | 8.1 | 17.8 | 34.6 | 37.4 | 26.0 | 22.1 | 38.0 |
| 24 h | N | 6 | 6 | 6 | 6 | 6 | 6 | 6 | 6 | 6 | 6 | 6 | 6 |
| | Mean | 6402.50 | 102.58 | 134.58 | 368.58 | 213.83 | 306.75 | 1645.83 | 1001.67 | 4001.67 | 347.58 | 1458.33 | 5.68 |
| | SD | 1473.82 | 17.63 | 37.30 | 70.50 | 26.80 | 54.78 | 324.61 | 201.14 | 1542.86 | 79.95 | 404.01 | 7.37 |
| | CV % | 23.0 | 17.2 | 27.7 | 19.1 | 12.5 | 17.9 | 19.7 | 20.1 | 38.6 | 23.0 | 27.7 | 129.7 |
| 96 h | N | 6 | 6 | 6 | 6 | 6 | 6 | 6 | 6 | 6 | 5 | 6 | 5 |
| | Mean | 5284.17 | 6.88 | 8.19 | 8.95 | 14.58 | 18.26 | 1554.17 | 58.44 | 970.00 | 8.63 | 1471.67 | 2.16 |
| | SD | 1628.39 | 1.12 | 1.99 | 2.43 | 5.74 | 3.59 | 162.09 | 16.62 | 465.60 | 2.22 | 403.62 | 0.14 |
| | CV % | 30.8 | 16.2 | 24.2 | 27.1 | 39.4 | 19.6 | 10.4 | 28.4 | 48.0 | 25.8 | 27.4 | 6.3 |

Note:
The unit of tissue concentration is (ng/g), and the unit of plasma concentration is (ng/mL).

The above results indicated that the distribution of LN003 in tumor-bearing mice after injection was tumor>lymph node>stomach>testis>kidney>spleen>duodenum>lung>liver>heart>plasma>brain, while after administration of the control JEVTANA solution, the drug distribution in tissues was tumor>lymph node>brain>testis>stomach>kidney>spleen>duodenum>lung>liver>heart>plasma.

The distribution of Cabazitaxel in tissues such as the heart and liver after administration of Cabazitaxel micelles was significantly less than that after administration of the control JEVTANA solution, with the lowest distribution in brain tissue; the peak drug concentration was only 11.6% of that of the control JEVTANA solution, showing a statistically very significant difference.

The test results suggested that Cabazitaxel micelle, adopting a special polymer micelle technology, significantly reduce the amount of Cabazitaxel crossing the blood-brain barrier and were expected to reduce neurotoxicity.

### Example 8: Cabazitaxel Micelle Allergy Hemolysis Test

### 8.1 Cabazitaxel Micelle: Hemolysis Test

In this test, blood was collected from New Zealand rabbits and prepared as a 2% red blood cell suspension. 0.9% sodium chloride injection (negative control), sterile water for injection (positive control), the test sample (the test sample was prepared from the Cabazitaxel micelle obtained by freeze-drying in Example 1 and stored in a borosilicate glass vial, and was diluted with 0.9% sodium chloride injection to concentrations of 0.5 mg/mL and 1 mg/mL), and the excipient sample (PEG2000-DSPE) were added separately. After thorough mixing, the samples were placed in a 37° C. biochemical incubator. The solution appearance was observed at 15, 30, 45, 60, 120, and 180 minutes after addition to determine whether hemolysis or red blood cell aggregation occurred. After direct observation, the solution in the test tubes was transferred to dry centrifuge tubes and centrifuged at 1500 rpm at room temperature for 10 minutes. The absorbance of the supernatant was measured at 540 nm using a spectrophotometer (with 0.9% sodium chloride injection as the blank), and the hemolysis rate was calculated.

The visual observation results showed that no hemolysis or aggregation occurred in the negative control tube (0.9% sodium chloride injection), only hemolysis was observed in the positive control tube (sterile water for injection), and no hemolysis or aggregation was observed in the test sample tubes (0.5 mg/mL and 1 mg/mL) and the excipient sample tube (PEG2000-DSPE). The spectrophotometric detection results showed that the hemolysis rates of the test sample tubes (0.5 mg/mL and 1 mg/mL) and the excipient sample tube were less than 5%, indicating no hemolysis. In conclusion, under the conditions of this test, the Cabazitaxel micelle at concentrations of 0.5 mg/mL and 1 mg/mL did not cause hemolysis or aggregation in the 2% New Zealand rabbits red blood cell suspension.

### 8.2 Cabazitaxel Micelle: Guinea Pig Active Systemic Anaphylaxis Test

In this test, 32 guinea pigs, half male and half female, were randomly divided into 4 groups, with 4 females and 4 males in each group. The negative control group was administered 5% glucose injection. The positive control group was administered ovalbumin (dissolved in 5% glucose injection) at a sensitization dose of 5 mg/kg. The low-dose group and high-dose group were administered Cabazitaxel micelle (prepared from the lyophilized sample of Example 1 stored in a borosilicate glass vial, and formulated with 5% glucose injection to the required concentrations for administration) at sensitization doses of 0.15 mg/kg and 0.3 mg/kg, respectively. Sensitization was performed by intraperitoneal injection at a sensitization volume of 1.5 mL/kg, once every other day, 3 times in total. On Days 14 and 21 after the last sensitization, the test or control sample was injected intravenously at twice the sensitization volume to challenge the animals, and the allergic reactions in guinea pigs were observed. The test results are as follows:
Clinical observation: Except for mild weight loss in animals of the high-dose group on Day 9, no other abnormal conditions were observed in the remaining animals during clinical observation.
Body weight: Compared with the negative control group, a statistically significant differences were observed in the body weight of male animals in the high-dose test sample group after the first challenge (P≤0.05), and in the body weight of female animals in the high-dose test sample group after the first challenge (P≤0.01). No other significant abnormalities were observed in the remaining animals.
Observation of symptoms after challenge: Animals in the positive control group began to show allergic symptoms 12 to 49 seconds after challenge and died within 2 to 5 minutes. The allergic reactions were all extremely positive, with an allergic reaction rate of 100%. No allergic reaction symptoms were observed in the negative control group low-dose group, and high-dose group after challenge.

In conclusion, under the conditions of this test, the active systemic anaphylaxis reaction in guinea pigs was negative when Cabazitaxel micelles were administered at the sensitization doses of 0.15 mg/kg and 0.3 mg/kg.

### 8.3 Cabazitaxel Micelles: Guinea Pig Passive Cutaneous Anaphylaxis Test

The preparation method of the low-dose group and high-dose group of Cabazitaxel micelles: The freeze-dried formulation samples stored in neutral borosilicate glass vials from Example 1 were taken as raw materials and reconstituted to the required administration concentration using glucose injection.

In this experiment, 40 guinea pigs were used, including 8 females and 8 males for the preparation of sensitization serum. They were evenly divided into 4 groups, with an equal number of females and males in each group: namely, the negative control group (glucose injection), the positive control group (ovalbumin, sensitization dose of 5 mg/kg), the low-dose group (Cabazitaxel micelle, sensitization dose of 0.15 mg/kg), and the high-dose group (Cabazitaxel micelle, sensitization dose of 0.3 mg/kg). Intraperitoneal injection was employed for sensitization at a dose volume of 1.5 mL/kg, once every other day for a total of 3 times. On the 14th day after the last sensitization, the animals were anesthetized, and blood was collected from the abdominal aorta to prepare serum, which was stored at −20° C. for subsequent use. Another 12 females and 12 males were used for intradermal sensitization, and they were also evenly divided into 4 groups, namely the negative control group, the low-dose group, the high-dose group, and the positive control group, with an equal number of females and males in each group. First, the corresponding sensitization serum was administered according to the group, with dilution ratios of 1:2, 1:4, and 1:8 (the amount of serum to be added was calculated based on the administration dose). 47 hours later, the challenge solution (a mixture of the test substance at the same sensitization dose as the corresponding group and 1% Evans blue solution in equal volume) was intravenously injected, with a dosage volume twice that of the intraperitoneal injection for sensitization. 26 to 34 minutes after the challenge, the guinea pigs were euthanized with CO2, and the skin on the back of the guinea pigs was incised to measure the size of the blue spot on the inner side.
Clinical observation and body weight: Only the animals in the high-dose group exhibited mild weight loss. No abnormalities were observed in the clinical signs or body weights of the other animals.
Challenge results: The measurement results of the reaction spots after the challenge showed that obvious blue spots were observed in all animals of the positive control group, with diameters ranging from 14.61 mm to 51.55 mm, and the results were positive. No blue spots were formed at any serum dilution in the animals of the negative control group, the low-dose group, and the high-dose group of the test samples, and the results were negative.

In conclusion, under the conditions of this experiment, when the administration doses of Cabazitaxel micelle were 0.15 mg/kg and 0.3 mg/kg, the passive cutaneous anaphylaxis reaction in guinea pigs was negative.

In the following examples, unless otherwise specified, “Cabazitaxel micelle prepared by spray drying” all refer to the Cabazitaxel micelle obtained by spray drying and hydration as described in Example 4. “Cabazitaxel micelle prepared by film hydration” all refer to the Cabazitaxel micelle obtained by film hydration as described in Example 1.

### Example 9

### (1) Reconstitution Stability

After dialysis at 37° C. for 4 hours, a large amount of flaky drug precipitate was found in the dialysis bag of the Cabazitaxel micellar formulation prepared by the film hydration method, while no drug was detected in the dialysis fluid. This indicates that when CTX-TW80 was released, the local drug concentration was much higher than the solubility of the drug in the dialysis fluid, leading to drug crystallization and precipitation. Therefore, this method is not suitable for investigating the release behavior of CTX-TW80 formulations. The states of the micelle prepared by the two methods in the dialysis bag after 4 hours of release at 37° C. are shown in FIGS. 28 and 29. Specifically, FIG. 28 shows the state of the micelle prepared by the film hydration method after 4 hours of release at 37° C. in the dialysis bag, and FIG. 29 shows the state of the micelle prepared by the spray drying method under the same release conditions.

A comparison of the states of the Cabazitaxel micelle prepared by the film hydration method and the spray drying method in the dialysis bag after 4 hours of dissolution at 37° C., demonstrates that the stability of the spray-dried micelle is significantly higher than that of the film-hydrated micelle.

### (2) Dilution Stability

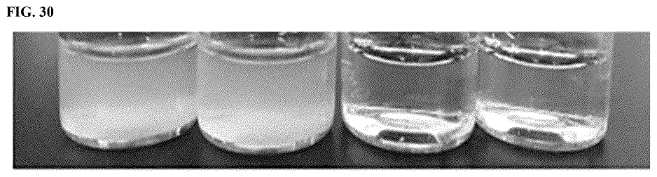

The Cabazitaxel micelle prepared by the spray drying method and the film hydration method were respectively diluted with 5% human serum albumin (5% HSA) and deionized water to prepare micelle sample solutions with a concentration of 1 mg/mL. Their states after 8 hours of storage at 37° C. are shown in FIG. 30.

FIG. 30 shows the states of the micelle after 8 hours of storage at 37° C. in different dilution media. From left to right, the samples are as follows: the micelle sample solution of the film-hydrated micelle diluted with 5% human serum albumin, the micelle sample solution obtained by diluting the micelle prepared by the film-hydration method with deionized water, the micelle sample solution of the spray-dried micelle diluted with 5% human serum albumin, and the micelle sample solution of the spray-dried micelle diluted with deionized water.

The above results indicate that the micelle prepared by the spray drying method exhibit better stability regardless of whether they are in deionized water or 5% HSA. After 8 hours of storage at 37° C., the micelle solution remains clear and transparent, indicating that no drug has crystallized or been released from the micelle. In contrast, for the micelle prepared by the film hydration method, drug precipitation occurred and the solution became turbid after 6 hours of storage at 37° C. in deionized water. In the 5% albumin solution, obvious precipitation was observed in the film-hydrated micelle after 6 hours of storage, while the spray-dried micelle remained clear and transparent. This indicates that the micelle prepared by the spray drying method have a stable structure and stronger dilution resistance compared with those prepared by the film hydration method.

### Example 10

Comparison of the biological activity of micelles prepared by spray drying and film hydration methods.

### (1) Pharmacodynamics Comparison

The preparation methods of the test Cabazitaxel micelles by the two methods:
Spray drying method: Under sterile conditions, the freeze-dried formulation samples stored in neutral borosilicate glass vials from Example 1 are taken as the test samples. Each vial of the test sample is dispersed and dissolved in an appropriate volume of 5% glucose solution. Shake the vial gently to completely disperse and dissolve the freeze-dried powder. There is some foam in the vial under negative pressure, and the foam subsides after air is injected to balance the pressure. After preparation, the drug solution is kept at room temperature for 30 minutes to ensure complete dissolution of the drug. The drug solution is then diluted with 5% glucose solution to achieve Cabazitaxel concentrations of 7.5 mg/mL, 3.75 mg/mL, and 1.875 mg/mL in the solution.
Film hydration method: The freeze-dried products prepared by the film hydration method in Example 1 are formulated into solutions with Cabazitaxel concentrations of 7.5 mg/mL and 3.75 mg/mL by the above method.

When the tumor grew to an average of about 158 mm³, 60 male BALB/c nude mice with appropriate tumor sizes were randomly divided into 6 groups, 10 mice in each group, namely the solvent control group G1 (5% glucose, 0 mg/kg), Cabazitaxel micelle prepared by the film hydration method (7.5 mg/kg, G2; 3.75 mg/kg, G3), and Cabazitaxel micelle prepared by the spray drying method (7.5 mg/kg, G4; 3.75 mg/kg, G5; 1.875 mg/kg, G6). All mice were administered intravenously once a week for 3 weeks.

Compared with the solvent control group, the Cabazitaxel micelle prepared by the film hydration method significantly inhibited the growth of subcutaneous xenograft tumors of prostate cancer PC-3, exhibiting a significant decrease in TV (tumor volume) and RTV (relative tumor volume) starting from D4. The RTV values of the 7.5 mg/kg and 3.75 mg/kg dose groups of Cabazitaxel micelle prepared by the film hydration method on D21 were 1.88 and 4.49, respectively, with relative tumor inhibition rates of 82.00% and 57.12%, and tumor weight inhibition rates of 73.60% and 51.69%, respectively.

Compared with the solvent control group, the Cabazitaxel micelle prepared by the spray drying method significantly inhibited the growth of subcutaneous xenograft tumors of prostate cancer PC-3, exhibiting a significant decrease in TV and RTV starting from D4, and the tumor inhibition effect was dose-dependent. The RTV values of the 7.5 mg/kg, 3.75 mg/kg, and 1.875 mg/kg dose groups of Cabazitaxel micelle prepared by the spray drying method on D21 were 1.53, 3.37, and 6.26, respectively, with relative tumor inhibition rates of 85.40%, 67.82%, and 40.25%, and tumor weight inhibition rates of 81.46%, 62.92%, and 29.21%, respectively.

The above experimental results indicate that at the same dose, especially at 3.75 mg/kg, the Cabazitaxel micelle prepared by the spray drying method exhibit better tumor inhibition effects than those prepared by the film hydration method, and there are significant differences in their tumor inhibition effects.

Relative tumor inhibition rate TGI (%)=(1−average RTV of the treatment group (T)/average RTV of the negative control group (C))×100%.

The average relative tumor volume of each group at each time point is shown in Table 12, the relative tumor inhibition rate (%) of each group at each time point is shown in Table 13.

TABLE 12
The relative tumor volume (average) of each group at each time point

| Time | G1 | G2 | G3 | G4 | G5 | G6 |
|---|---|---|---|---|---|---|
| (day) | (5% glucose) | 7.5 mg/kg | 3.75 mg/kg | 7.5 mg/kg | 3.75 mg/kg | 1.875 mg/kg |
| 0 | 1.00 | 1.00 | 1.00 | 1.00 | 1.00 | 1.00 |
| 4 | 1.65 | 1.13 | 1.15 | 1.10 | 1.17 | 1.34 |
| 7 | 2.78 | 0.99 | 0.98 | 0.87 | 1.02 | 1.60 |
| 10 | 3.77 | 0.96 | 1.32 | 0.71 | 0.98 | 2.1 |
| 12 | 5.17 | 0.87 | 1.53 | 0.68 | 1.15 | 2.67 |
| 14 | 6.04 | 0.84 | 1.89 | 0.67 | 1.42 | 3.31 |
| 17 | 7.66 | 1.16 | 2.54 | 0.75 | 1.77 | 4.29 |
| 19 | 8.82 | 1.54 | 3.26 | 0.97 | 2.67 | 5.23 |
| 21 | 10.47 | 1.88 | 4.49 | 1.53 | 3.37 | 6.26 |

TABLE 13
The relative tumor inhibition rate (%) of each group at each time point

| | G2 | G3 | G4 | G5 | G6 |
|---|---|---|---|---|---|
| Time (day) | 7.5 mg/kg | 3.75 mg/kg | 7.5 mg/kg | 3.75 mg/kg | 1.875 mg/kg |
| 4 | 31.56 | 30.58 | 33.25 | 29.48 | 19.08 |
| 7 | 64.25 | 64.72 | 68.75 | 63.22 | 42.31 |
| 10 | 74.46 | 64.93 | 81.09 | 74.14 | 44.25 |
| 12 | 83.17 | 70.48 | 86.79 | 77.75 | 48.42 |
| 14 | 86.11 | 68.65 | 88.93 | 76.46 | 45.28 |
| 17 | 84.90 | 66.80 | 90.26 | 76.95 | 43.98 |
| 19 | 82.50 | 62.98 | 88.94 | 69.77 | 40.68 |
| 21 | 82.00 | 57.12 | 85.40 | 67.82 | 40.25 |

### (2) Pharmacokinetics in Rats

Sixteen SD rats were randomly divided into two groups (8 rats per group, with equal numbers of males and females), and each group was given a single intravenous injection of 3 mg/kg of Cabazitaxel micelle prepared by spray drying and 3 mg/kg of Cabazitaxel micelle prepared by film hydration, respectively. The changes in plasma drug concentration over time after a single intravenous administration of Cabazitaxel micelle prepared by spray drying and film hydration in SD rats were shown in Table 14.

TABLE 14
Drug concentration over time after a single intravenous
administration of Cabazitaxel micelles prepared
by spray drying and film hydration in SD rats

| | Spray drying (3 mg/kg) | | Film hydration (3 mg/kg) | |
|---|---|---|---|---|
| | Mean of | | Mean of | |
| Time (h) | Conc. (ng/ml) | SD | Conc. (ng/ml) | SD |
| 0 | 0 | 0 | 0 | 0 |
| 0.033 | 1058.7 | 283.87 | 1568.52 | 153.42 |
| 0.083 | 600.14 | 200.69 | 854.56 | 127.98 |
| 0.25 | 274.07 | 111.11 | 423.88 | 53.6 |
| 0.5 | 133.24 | 38.65 | 219.54 | 31.66 |
| 0.75 | 98.53 | 28.23 | 133.89 | 24.17 |
| 1 | 54.49 | 13.35 | 79.03 | 23.07 |
| 2 | 27.76 | 6.14 | 27.66 | 8.29 |
| 4 | 12.09 | 2.77 | 9.56 | 3.94 |
| 8 | 13.46 | 3.2 | 12.8 | 2.83 |
| 24 | 3.37 | 1.25 | 2.91 | 1.24 |
| 32 | 1.99 | 0.59 | 2.33 | 2.2 |

After SD rats were administered a single dose of 3 mg/kg of Cabazitaxel micelle prepared by spray drying, the maximum plasma concentration (Cₘₐₓ) was 1058.7 ng/mL, and the area under the plasma concentration-time curve (AUC) was 489.29 h*ng/mL. After SD rats were administered a single dose of 3 mg/kg of Cabazitaxel micelle prepared by film hydration, the Cₘₐₓ was 1568.52 ng/mL, and the AUC was 590.13 h*ng/mL. Compared with the Cabazitaxel micelle prepared by spray drying at 3 mg/kg, the Cₘₐₓ and AUC of the Cabazitaxel micelle prepared by film hydration were 1.48 times and 1.21 times as high, respectively. The exposure (Cₘₐₓ and AUC) of the Cabazitaxel micelle prepared by spray drying was significantly lower than that of the Cabazitaxel micelle prepared by film hydration (p<0.05). The above results indicate that the exposure (Cₘₐₓ and AUC) of the Cabazitaxel micelle prepared by spray drying was significantly lower than that of the Cabazitaxel micelle prepared by film hydration (p<0.05).

These results indicated that the exposure (Cmax and AUC) of Cabazitaxel micelle prepared by the spray-drying method was significantly lower than that prepared by the film-hydration method (p<0.05). However, according to the pharmacodynamic comparison in (1), the therapeutic effect of the micelle prepared by spray drying was better at the same dose, this suggested that even though the AUC of the Cabazitaxel micelle prepared by spray drying is lower, they still have a better therapeutic effect under the safety advantage brought by the lower AUC due to their superior targeting capability. Moreover, due to the lower AUC and better safety, the Cabazitaxel micelle prepared by spray drying are expected to increase the clinical efficacy by increasing the dosage while ensuring safety.

### (3) Acute Toxicity Test

Single-Dose Intravenous Injection of SD Rats with 21-Day Recovery Toxicity Test

Thirty SD rats, half male and half female, were randomly divided into three groups, including the solvent control group (5% glucose injection), the spray-dried group 15 mg/kg, and the film-hydrated group 15 mg/kg. Five rats (gender group) (the meaning of “gender group” was each gender, each group; for example, 5 rats (gender group) meant each dosing group contained 5 male animals and 5 female animals), with a dosing volume of 5 mL/kg, and a single tail vein injection followed by a 21-day drug-free recovery period.

In the 15 mg/kg spray-dried Cabazitaxel micelle group, a decrease in white blood cell count (WBC), lymphocyte count (#LYMPH), neutrophil count (#NEUT), monocyte count (#MONO), eosinophil count (#EOS), large unstained cell count (#LUC), platelet count (PLT), and reticulocyte count (#RETIC) was observed on day 3, with an increase in mean platelet volume (MPV). Among them, the decrease rate of #NEUT in male SD rats was −30.0%, and that in female SD rats was −19.5%. In the 15 mg/kg film-hydrated Cabazitaxel micelle group, the above indicators also changed on day 3, with the decrease rates of #NEUT in male and female SD rats being −49.2% and −41.4%, respectively, both higher than those of the spray-dried Cabazitaxel micelle at the same dose. Moreover, in female SD rats, a decrease in #NEUT was still observed on day 22 after administration of 15 mg/kg film-hydrated Cabazitaxel micelle. At the same dose, the spray-dried Cabazitaxel micelle had a better effect on reducing neutrophils and recovery compared to the film-hydrated Cabazitaxel micelle, suggesting that the spray-dried Cabazitaxel micelle for injection might improve the impact on neutrophil reduction and had better safety in clinical use.

The maximum tolerated dose (MTD) of spray-dried Cabazitaxel micelle was 15 mg/kg, with the main toxicities being sparse hair, decreased body weight and average food intake, and changes in hematology and the prostate. At the same dose, the 15 mg/kg film-hydrated Cabazitaxel micelle group also showed changes in body weight, average food intake, and hematology, which were basically consistent with the 15 mg/kg spray-dried Cabazitaxel micelle group. In addition, the film-hydrated Cabazitaxel micelle group also showed symptoms such as fluffed fur, soiled perianal area/diarrhea, head swelling, and hair loss, as well as histopathological changes in the epididymis, nictitating membrane, bladder, and testis, while the spray-dried Cabazitaxel micelle did not show these symptoms.

It could be seen that at the same dose, the spray-dried Cabazitaxel micelle formulation was safer than the film-hydrated Cabazitaxel micelle formulation.

Although the above description sets forth specific embodiments of the present invention, it should be understood by those skilled in the art that these are merely illustrative examples. Under the premise of not departing from the principles and essence of the present invention, various changes or modifications can be made to these embodiments. Therefore, the scope of protection of the present invention is defined by the appended claims.

1-13. (canceled)

14. Cabazitaxel-containing micelles, wherein the cabazitaxel-containing micelles comprise cabazitaxel and polyethylene glycol 2000 distearoyl phosphatidylethanolamine; and wherein the polydispersity index of the cabazitaxel-containing micelles is no more than 0.09.

15. The cabazitaxel-containing micelles according to claim 14, wherein the polydispersity index of the cabazitaxel-containing micelles is no more than 0.08.

16. The cabazitaxel-containing micelles according to claim 14, wherein particle size of the cabazitaxel-containing micelles is about 10-20 nm.

17. The cabazitaxel-containing micelles according to claim 14, wherein encapsulation efficiency of the cabazitaxel-containing micelles is 95% to 100%.

18. The cabazitaxel-containing micelles according to claim 14, wherein encapsulation efficiency of the cabazitaxel-containing micelles is 98.5% to 100%.

19. The cabazitaxel-containing micelles according to claim 14, wherein total impurity content in the cabazitaxel-containing micelles is no more than 2%.

20. The cabazitaxel-containing micelles according to claim 14, wherein total impurity content in the cabazitaxel-containing micelles is no more than 1%.

21. The cabazitaxel-containing micelles according to claim 14, wherein total impurity content in the cabazitaxel-containing micelles is no more than 0.1%.

22. The cabazitaxel-containing micelles according to claim 14, wherein Zeta potential of the cabazitaxel-containing micelles is (−45 mv) to (−60 mv).

23. The cabazitaxel-containing micelles according to claim 14, wherein Zeta potential of the cabazitaxel-containing micelles is (−45 mv) to (−50 mv).

24. The cabazitaxel-containing micelles according to claim 14, wherein mass ratio of the polyethylene glycol 2000 distearoyl phosphatidylethanolamine to the cabazitaxel is no more than 15:1.

25. The cabazitaxel-containing micelles according to claim 14, wherein mass ratio of the polyethylene glycol 2000 distearoyl phosphatidylethanolamine to the cabazitaxel is (5-15):1.

26. The cabazitaxel-containing micelles according to claim 14, wherein mass ratio of the polyethylene glycol 2000 distearoyl phosphatidylethanolamine to the cabazitaxel is about 11:1.

27. The cabazitaxel-containing micelles according to claim 14, wherein the cabazitaxel-containing micelles satisfy at least one of the following conditions:
(i) the polydispersity index of the cabazitaxel-containing micelles is about 0.06;
(ii) the particle size of the cabazitaxel-containing micelles is about 15.0 nm;
(iii) the encapsulation efficiency of the cabazitaxel-containing micelles is about 99.7%; and
(iv) the Zeta potential of the cabazitaxel-containing micelles is about −48.77 mv.

28. The cabazitaxel-containing micelle according to claim 26, wherein the cabazitaxel-containing micelles satisfy at least one of the following conditions:
(i) the polydispersity index of the cabazitaxel-containing micelles is about 0.06;
(ii) the particle size of the cabazitaxel-containing micelle is about 15.0 nm;
(iii) the encapsulation efficiency of the cabazitaxel-containing micelles is about 99.7%; and
(iv) the Zeta potential of the cabazitaxel-containing micelle is about −48.77 mv.

29. The cabazitaxel-containing micelles according to claim 14, wherein the cabazitaxel-containing micelles comprise:
about 60 mg of cabazitaxel; and
about 660 mg of polyethylene glycol 2000 distearoyl phosphatidylethanolamine.

30. The cabazitaxel-containing micelles according to claim 14, wherein the cabazitaxel-containing micelles further comprises one or more of the following components: C₁₋₆ alcohol solvents, water, glucose injection, and sodium chloride injection.

31. The cabazitaxel-containing micelles according to claim 14, wherein the cabazitaxel-containing micelles further comprises sterile water for injection, 5% glucose injection, or 0.9% sodium chloride injection.

32. The cabazitaxel-containing micelles according to claim 14, wherein the cabazitaxel-containing micelles satisfy at least one of the following conditions:
(i) reconstitution time of the cabazitaxel-containing micelles is no more than 60 minutes;
(ii) the release rate of the cabazitaxel-containing micelles is 75% to 100%;
(iii) the cabazitaxel-containing micelles are stably stored at room temperature for more than 6 hours; and
(iv) the cabazitaxel-containing micelles is stably stored at 4° C. for more than 24 hours.
